(19) United States
(12) Patent Application Publication
(10) Pub. No.: US 2009/0275557 A1
(43) Pub. Date: Nov. 5, 2009

(54) Novel Composition for Treatment of Diseases Related to Activated Lymphocytes

(75) Inventors: Stephen Roth, Gladwyne, PA (US); Clayton Buck, Berwyn, PA (US); Christopher Self, West Caldwell, NJ (US); Gary Olson, Mountainside, NJ (US); Nallaganchu Rao, Hillsborough, NJ (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Immune Control, Inc., Conshohocken, PA (US)

(21) Appl. No.: 12/393,888

(22) Filed: Feb. 26, 2009

Related U.S. Application Data
(60) Provisional application No. 61/067,695, filed on Feb. 29, 2008.

(51) Int. Cl. A61K 31/397; C07D 417/06; C12N 5/00; A61K 31/5415; A61P 11/06; A61P 19/02; C07D 417/14

(52) U.S. Cl. 514/210.21; 544/42; 435/377; 514/225.5

(57) ABSTRACT

The present invention relates to inhibiting proliferation and inducing apoptosis in activated lymphocytes, including T cells and B cells. The invention also provides compositions and methods for inhibiting proliferation and inducing apoptosis in activated lymphocytes, as well methods for treating diseases associated with activated lymphocytes by administering 5-HT receptor antagonists.

### BACKGROUND OF THE INVENTION

Serotonin (also referred to as 5-hydroxytryptamine or 5-HT) is a neurotransmitter that has been strongly implicated in the pathophysiology and treatment of a wide variety of neuropsychiatric disorders. Serotonin exerts its effects through a diverse family of serotonin receptor molecules (referred to herein as “5-HT receptors” or “5-HTRs”). Classically, members of the serotonin receptor family have been grouped into seven (7) subtypes pharmacologically, i.e., according to their specificity of various serotonin antagonists. Thus, while all the 5-HT receptors specifically bind with serotonin, they are pharmacologically distinct and are encoded by separate genes. To date, fourteen (14) mammalian serotonin receptors have been identified and sequenced. More particularly, these fourteen separate 5-HT receptors have been grouped into seven (7) pharmacological subtypes, designated 5-HT1, 5-HT2, 5-HT3, 5-HT4, 5-HT5, 5-HT6, and 5-HT7. Several of the subtypes are further subdivided such that the receptors are grouped pharmacologically as follows: 5-HT1A, 5-HT1B, 5-HT1D, 5-HT1E, 5-HT1F, 5-HT2A, 5-HT2B, 5-HT2C, 5-HT3A, 5-HT3B, 5-HT4, 5-HT5A, 5-HT6, 5-HT7. However, when the nucleic and amino acid sequences of the receptors are compared, the percent identity among the subtypes is not correlated to the pharmacological groupings.

Of the fourteen different mammalian serotonin receptors that have been cloned, all but one are members of the G-protein coupled receptor superfamily. Serotonin receptors 5-HT1A, 5-HT1B, and 5-HT1D inhibit adenylate cyclase, and 5-HT2 receptors activate phospholipase C pathways, stimulating breakdown of polyphosphoinositides. The 5-HT2 receptor belongs to the family of rhodopsin-like signal transducers that are distinguished by a seven-transmembrane configuration and functional linkage to G-proteins. The 5-HT3 receptor family includes ligand-gated ion channel receptors that have four putative TMDs.

Serotonin regulates a wide variety of sensory, motor and behavioral functions in the mammalian CNS, including behaviors such as learning and memory, sleep, thermoregulation, motor activity, pain, sexual and aggressive behaviors, appetite, neuroendocrine regulation, and biological rhythms. Serotonin has also been linked to pathophysiological conditions such as anxiety, depression, obsessive-compulsive disorders, schizophrenia, suicide, autism, migraine, emesis, alcoholism and neurodegenerative disorders. This biogenic amine neurotransmitter is synthesized by neurons of the brain stem that project throughout the CNS, with highest density in basal ganglia and limbic structures (Steinbusch, 1984, In: Handbook of Chemical Neuroanatomy 3:68-125, Bjorklund et al., Eds., Elsevier Science Publishers, B. V.).

Studies have suggested that serotonin may play a role in the immune system since data demonstrate that serotonin receptors are present on various cells of the immune system. There have been reports in the literature about the immunomodulatory effects of adding serotonin exogenously to mitogenically stimulated lymphocyte cultures. Under some circumstances, serotonin has been shown to stimulate the activated T cells (Foon et al., 1976, J. Immunol. 117:1545-1552; Kut et al., 1992, Immunopharmacol. Immunotoxicol. 14:783-796; Young et al., 1993, Immunology 80:395-400), whereas other laboratories report that high concentrations of added serotonin inhibit the proliferation (Slauson et al., 1984, Cell. Immunol. 84:240-252; Khan et al., 1986, Int. Arch. Allergy Appl. Immunol. 81:378-380; Mossner & Lesch, 1998, Brain, Behavior, and Immunity 12:249-271).

Of the fourteen known pharmacologically distinct serotonin receptors, lymphocytes express type 2A, type 2B, type 2C, type 6 and type 7 on resting cells (Ameisen et al., 1989, J. Immunol. 142:3171-3179; Stefulj et al., 2000, Brain, Behavior, and Immunity 14:219-224) and that the type 1A and type 3 receptors are up-regulated upon activation (Aune et al., 1993, J. Immunol. 151:1175-1183; Meyniel et al., 1997, Immunol. Lett. 55:151-160; Stefulj et al., 2000, Brain, Behavior, and Immunity 14:219-224).

The involvement of the 5-HT1A receptors in human and murine T cells has also been demonstrated (Aune et al., 1990, J. Immunol. 145:1826-1831; Aune et al., 1993, J. Immunol. 151:1175-1183; Aune et al., 1994, J. Immunol. 153:1826-1831). These studies established that IL-2-stimulated human T cell proliferation could be inhibited by a blockade of tryptophan hydroxylase, i.e., the first enzyme involved in the conversion of tryptophan to serotonin, and that the inhibition could be reversed by the addition of 5-hydroxy tryptophan. Furthermore, human T cell proliferation was blocked in vitro with a 5-HT1A-specific receptor antagonist. In a murine model, a type 1A receptor antagonist, but not a type 2 receptor antagonist, was able to inhibit the in vivo contact sensitivity response, but not antibody responses, to oxazalone.

PCT Publication No. WO 03/106660 discloses the use of fluphenazine, an antagonist of 5-HT(1B/1D) and 5-HT(2C) receptors, for inhibiting proliferation and inducing cell death in lymphocytes.

There exists a long-felt need to develop novel compounds and therapies for treating diseases related to activated lymphocytes and lymphocyte proliferation, especially diseases related to activated T cells and B cells. In addition, there is a long-felt need to develop novel compounds without the side effects related to other serotonin receptor antagonists. The present invention meets these needs.

### BRIEF SUMMARY OF THE INVENTION

The present invention includes a compound of formula I

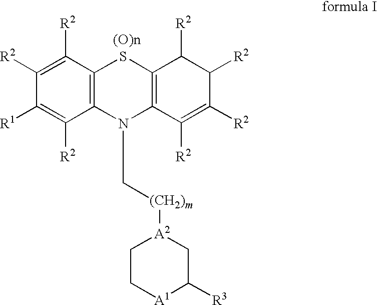

or a pharmaceutically acceptable salt, prodrug or solvate thereof, wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

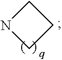

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

In one embodiment of the invention, R¹ is hydrogen, halogen, (C₁-C₆)alkyl, methyl, C≡N, C(═O)OR⁷, C(═O)NR⁷₂, C(═O)NH₂, SO₂NR⁷₂, SO₂NMe₂, (C₁-C₃)perfluoroalkyl, or CF₃.

In another embodiment of the invention, each occurrence of R² is hydrogen.

In another embodiment, R³ is hydrogen.

In another embodiment, A² is CH.

In another embodiment, R⁴ is (CH₂)ₚR⁵.

In another embodiment, R⁴ is C(═O)(CH₂)ₚR⁵.

In another embodiment, R⁴ is C(═O)(CH₂)ₚNR⁶₂.

In another embodiment of the invention, wherein R⁴ is C(═O)(CH₂)ₚR⁵, m is 2, n is 0, p is 2, and q is 1.

In another embodiment of the invention, wherein R⁴ is (CH₂)ₚR⁵, wherein m is 2, n is 0, p is 2, and q is 3.

In another embodiment, the compound is selected from the group consisting of ICI-1506, ICI-1520, ICI-1551, and ICI-1552.

The present invention also includes compounds selected from the group consisting of ICI-955, ICI-956, ICI-957, ICI-1247, ICI-1259, ICI-1260, ICI-1451, and ICI-505.

The invention further includes a method of inducing apoptosis in an immune cell. The method comprises contacting the immune cell with a composition comprising a compound of formula I or a composition comprising a compound selected from the group consisting of ICI-955, ICI-956, ICI-957, ICI-1247, ICI-1259, ICI-1260, ICI-1451, and ICI-1505; wherein a compound of formula I has the structure:

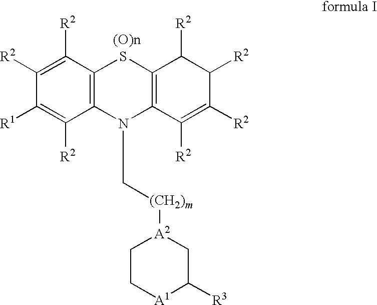

and wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

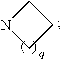

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

In certain embodiments, the immune cell is a lymphocyte.

In certain embodiments, the lymphocyte is selected from the group consisting of a T cell and a B cell.

In other embodiments, the B cell is a plasma cell.

In another embodiment, the plasma cell is a multiple myeloma cell.

The present invention also includes a method of inhibiting proliferation of a lymphocyte. The method comprises contacting the lymphocyte with a composition comprising a compound of formula I or a composition comprising a compound selected from the group consisting of ICI-955, ICI-956, ICI-957, ICI-1247, ICI-1259, ICI-1260, ICI-1451, and ICI-1505; wherein a compound of formula I has the structure:

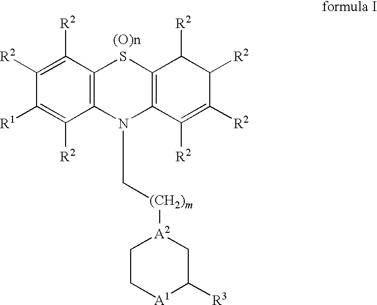

and wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

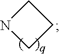

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

In one embodiment of the method, the lymphocyte is selected from the group consisting of a T cell and a B cell.

In another embodiment of the method, the B cell is a plasma cell.

In yet another embodiment, the plasma cell is a multiple myeloma cell.

The present invention also includes a method of treating a disease characterized by abnormal lymphocyte proliferation. The method comprises administering to a mammal a composition comprising a compound of formula I or a composition comprising a compound selected from the group consisting of ICI-955, ICI-956, ICI-957, ICI-1247, ICI-1259, ICI-1260, ICI-1451, and ICI-1505; wherein a compound of formula I has the structure:

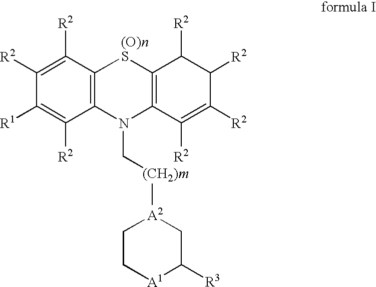

and wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

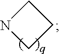

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

The present invention also includes a method of treating a disease selected from the group consisting of asthma and rheumatoid arthritis. The method comprises administering to a mammal a composition comprising a compound of formula I or a composition comprising a compound selected from the group consisting of ICI-955, ICI-956, ICI-957, ICI-1247, ICI-1259, ICI-1260, ICI-1451, and ICI-1505; wherein a compound of formula I has the structure:

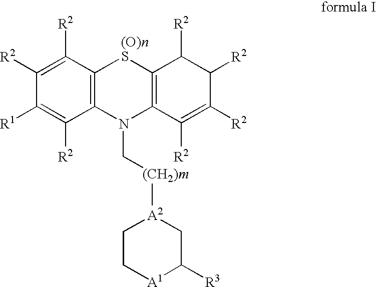

and wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

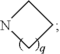

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

In certain embodiments of the method for treating a disease selected from the group consisting of asthma and rheumatoid arthritis wherein the method comprises administering to a mammal a composition comprising a compound of formula I or a composition comprising a compound selected from the group consisting of ICI-955, ICI-956, ICI-957, ICI-1247, ICI-1259, ICI-1260, ICI-1451, and ICI-1505, the mammal is a human.

### BRIEF DESCRIPTION OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a synthetic scheme for preparing ICI-955.

FIG. 2 is a synthetic scheme for preparing ICI-956 and ICI-957.

FIG. 3 is a synthetic scheme for preparing ICI-1247.

FIG. 4 is a synthetic scheme for preparing ICI-1259.

FIG. 5 is a synthetic scheme for preparing ICI-1260.

FIG. 6 is a synthetic scheme for preparing ICI-1451.

FIG. 7 is a synthetic scheme for preparing ICI-1505.

FIG. 8 is a synthetic scheme for preparing ICI-1506.

FIG. 9 is a synthetic scheme for preparing ICI-1520.

FIG. 10 is a synthetic scheme for preparing acid chloride 12.

FIG. 11 is a synthetic scheme for preparing ICI-1551.

FIG. 12 is a synthetic scheme for preparing acid chloride 14.

FIG. 13 is a synthetic scheme for preparing ICI-1552.

FIG. 14 is a graph showing the average plasma concentration of ICI-1259 versus time following oral administration in male Sprague-Dawley rats at 10 mg/kg.

FIG. 15 is a graph showing the average plasma concentration of ICI-1260 versus time following oral administration in male Sprague-Dawley rats at 10 mg/kg.

FIG. 16 is a graph depicting the oral efficacy of ICI-1259 in reducing ankle swelling in bovine-collagen immunized rats.

FIG. 17 is a graph depicting the oral efficacy of ICI-1260 in reducing ankle swelling in bovine-collagen immunized rats.

FIG. 18 is a graph depicting the efficacy of IP injected ICI-1259, ICI-1260, and ICI-1505 in reducing ankle swelling in bovine-collagen immunized rats.

FIG. 19 is a graph depicting the efficacy of IP injected ICI-1506 in reducing ankle swelling in bovine-collagen immunized rats.

FIG. 20 is a bar graph depicting the ability of ICI-1259 and ICI-1260 to bind to 5-HT1A(h), 5-HT1B, 5-HT1D(h), 5-HT2A, and 5-HT2A(h).

FIG. 21 is a bar graph depicting the ability of ICI-1259 and ICI-1260 to bind to 5-HT2B, 5-HT2C, 5-HT3, 5-HT3(h).

FIG. 22 is a graph depicting the ability of ICI-1259 and ICI-1260 to bind to 5-HT4, 5-HT5A(h), 5-HT6, and 5-HT7.

FIG. 23 is a graph depiciting the ability of ICI-1506 to inhibit 5-HT1A(h), 5-HT1B, 5-HT1D(h), 5-HT2A, 5-HT2A(h), 5-HT2B(h), 5-HT2C, 5-HT3, 5-HT3(h), 5-HT4, 5-HT5A(h), 5-HT6, and 5-HT7.

FIG. 24 is a graph depiciting the ability of ICI-1520 to inhibit 5-HT1A(h), 5-HT1B, 5-HT1D(h), 5-HT2A, 5-HT2A(h), 5-HT2B(h), 5-HT2C, 5-HT3, 5-HT3(h), 5-HT4, 5-HT5A(h), 5-HT6, and 5-HT7.

### DETAILED DESCRIPTION OF THE INVENTION

The present invention relates to compositions and methods for inducing cell death and/or apoptosis in activated lymphocytes. In addition, the present invention relates to compositions and methods for inhibiting the proliferation of activated lymphocytes. The novel serotonin receptor antagonists disclosed herein inhibit proliferation and induce apoptosis in various lymphocyte cell lines, including neoplastic T cells and B cells. Thus, the present invention encompasses methods, compositions and kits for inhibiting the proliferation of lymphocytes and for inducing apoptosis in lymphocytes. The compositions and methods of the present invention are useful for treating various diseases associated with the proliferation and/or activation of lymphocytes, including, but not limited to lymphomas, myelomas, autoimmune diseases, transplant rejection, and the like.

### DEFINITIONS

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

By T cell “activation,” as the term is used herein, is meant that the T cell, when contacted with a compound, molecule, or cell capable of generating an immune response (e.g., a mitogen or antigen), detectably upregulates surface markers, such as CD25, i.e., the IL-2 receptor, initiates a phosphorylation cascade involving p56lck, causes the release of cytokines and interleukins, increases DNA synthesis which can be assessed by, among other methods, assessing the level of incorporation of ³H-thymidine into nascent DNA strands, and causes the cells to proliferate.

A “serotonin antagonist” is a composition of matter which, when administered to a mammal such as a human, detectably inhibits a biological activity attributable to the level or presence of serotonin.

A “serotonin receptor antagonist” is a composition of matter which, when administered to a mammal such as a human, detectably inhibits a biological activity attributable to the of serotonin to a serotonin receptor.

By the term “selective antagonist,” as these terms are used herein, is meant a chemical agent that has at least about a 5-fold greater affinity for the target serotonin receptor type than for any other serotonin receptor family member.

As used herein, to “alleviate” a disease means reducing the severity of one or more symptoms of the disease.

By the term “allogeneic graft,” as used herein, is meant grafting of any tissue within a species wherein there is a mismatch of an immunological marker, such as, but not limited to, the major histocompatibility complex (MHC), and/or a minor antigen.

The term “allogeneic graft response”, as used herein, means any immune response directed against non-self tissue grafted into a recipient. Grafting procedures include, but are not limited to, administering non-self cells, tissue, or organs during, e.g., bone marrow transplantation, organ transplant, and the like.

The term “apoptosis,” as used herein, means an active process, involving the activation of a preexisting cellular pathway, induced by an extracellular or intracellular signal, causing the death of the cell. In particular, the cell death involves nuclear fragmentation, chromatin condensation, and the like, in a cell with an intact membrane.

By the term “applicator,” as the term is used herein, is meant any device including, but not limited to, a hypodermic syringe, a pipette, and the like, for administering the inhibitor of serotonin interaction with a serotonin receptor (e.g., a serotonin receptor antagonist) of the invention to a mammal.

A “cell cycle process,” as used herein, means any cellular function or process associated with the cell cycle and the various phases thereof. Thus, a cell cycle process is one associated with, or which mediates or is involved in, the cell progressing through any portion of the cell cycle.

Inhibition of serotonin signaling is “deleterious” to a cell, as the term is used herein, where the inhibition mediates a detectable decrease in the viability of the cell. Cell viability can be assessed using standard methods that are well-known in the art, including, but not limited to, assessing the level of biomolecular synthesis (e.g., protein synthesis, nucleic acid synthesis, and the like), trypan blue exclusion, MTT reduction, uptake of propidium iodide, exposure of phosphatidylserine on the cell surface, DNA fragmentation and/or ladder formation, and the like.

A “disease” is a state of health of an animal wherein the animal cannot maintain homeostasis, and wherein if the disease is not ameliorated, then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

By the term “does not substantially cross the blood-brain barrier”, as used herein, means that the inhibitor does not detectably cross the blood-brain barrier as assessed using standard assays such as those disclosed herein, known in the art, or such assays as are developed in the future to determine the permeability of a compound across the blood-brain barrier. Such assays include, but are not limited to, assessing the neuro-psychotropic effects of the compound when administered to an animal. Further, the assays encompass, among other things, assessing the concentration of the compound beyond the barrier, or an art-recognized model of the blood-brain barrier, over time to determine the permeability of the compound through the barrier.

It would be understood by the artisan that an inhibitor can be ab initio impermeable and not cross the blood-brain barrier at a detectable level. Further, it would be understood that an inhibitor of interest can be modified, using techniques well-known in the art, such that it does not detectably cross the blood-brain barrier, or crosses it at a detectably lower level that it did before it was modified. In both instances, whether it loses its ability to cross the blood-brain barrier at a detectable level or loses the ability to cross it at a lower level than before it was modified, the compound is considered to “not substantially cross the blood-brain barrier” for purposes of this section.

By the term “effective amount”, as used herein, is meant an amount of an inhibitor that is sufficient to mediate a detectable decrease in transmission of serotonin signaling via a serotonin receptor on a cell. Transmission of a serotonin signal can be assessed using standard methods well-known in the art, such as, but not limited to, those described elsewhere herein, including, for example, assessing the level of binding of serotonin with a receptor and/or assessing the level of activation of a cell.

The skilled artisan would understand that the amount varies and can be readily determined based on a number of factors such as the disease or condition being treated, the age and health and physical condition of the mammal being treated, the severity of the disease, the particular compound being administered, and the like. Generally, the dosage will be set between 0.01 mg/kg and 100 mg/kg. In one embodiment, the drug is administered through intravenous bolus injection. This type of bolus administration can be used to ensure that all of the immunologically relevant cells encounter sufficient quantity of the drug in order to block their receptor-mediated signals. However, the invention is not limited to this method of administration.

By the term “immune reaction,” as used herein, is meant the detectable result of stimulating and/or activating an immune cell.

“Immune response,” as the term is used herein, means a process that results in the activation and/or invocation of an effector function in either the T cells, B cells, natural killer (NK) cells, and/or antigen-presenting cells (APCs). Thus, an immune response, as would be understood by the skilled artisan, includes, but is not limited to, any detectable antigen-specific or allogeneic activation of a helper T cell or cytotoxic T cell response, production of antibodies, T cell-mediated activation of allergic reactions, and the like.

“Immune cell,” as the term is used herein, means any cell involved in the mounting of an immune response. Such cells include, but are not limited to, T cells, B cells, NK cells, antigen-presenting cells, and the like.

“Instructional material,” as that term is used herein, includes a publication, a recording, a diagram, or any other medium of expression which can be used to communicate the usefulness of the nucleic acid, peptide, and/or compound of the invention in the kit for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the nucleic acid, peptide, and/or compound of the invention or be shipped together with a container which contains the nucleic acid, peptide, and/or compound. Alternatively, the instructional material may be shipped separately from the container with the intention that the recipient uses the instructional material and the compound cooperatively.

By the term “serotonin family receptor” is meant any receptor which can be classified as a serotonin, adrenergic, histamine, melatonin, or dopaminergic receptor. That is, the receptor specifically binds with any of these molecules and does not significantly bind with other molecules in a sample.

A “serotonin receptor” includes a polypeptide that specifically binds with serotonin.

“Serotonin signal,” as the term is used herein, means a change in the balance of any intracellular biochemical pathway as a result of a receptor-mediated interaction with serotonin, a specific drug interaction with any serotonin-specific receptor, or both, that results in the change.

Similarly, “activation of a serotonin” receptor, as used herein, means that binding of serotonin with a serotonin receptor on a cell induces the typical cascade of intra and extracellular events associated with such binding.

A “receptor” is a compound that specifically binds with a ligand.

By the term “specifically binds,” as used herein, is meant a receptor which recognizes and binds serotonin family molecules present in a sample (i.e., dopaminergic proteins, adrenergic protein, histamines, melatonin, and serotonin), but does not substantially recognize or bind other molecules in the sample.

To “treat” a disease as the term is used herein, means to reduce the frequency of the disease or disorder reducing the frequency with which a symptom of the one or more symptoms disease or disorder is experienced by an animal.

### DESCRIPTION

The present invention relates to methods, compositions, and kits for treating diseases and conditions associated with the proliferation of activated lymphocytes and the diseases resulting from the activation of lymphocytes. The present invention encompasses methods for inhibiting and killing activated lymphocytes, compositions that inhibit and/or kill activated lymphocytes, compositions that inhibit the proliferation of activated lymphocytes, and kits for using the methods and compositions of the invention.

The compositions of the present invention include 5-HT receptor antagonists having the chemical formulae disclosed elsewhere herein. The compositions disclosed herein further comprise combinations of these 5-HT receptor antagonists with additional compositions for inhibiting and/or killing activated lymphocytes. The compositions of the present invention inhibit and/or kill activated lymphocytes by, among other things, inducing apoptosis and cell death in activated lymphocytes. In addition, the compounds of the present invention inhibit proliferation of lymphocytes, such as T cells and B cells, and are therefore useful in the treatment of diseases where activated and/or proliferating lymphocytes cause pathology. Such diseases include, but are not limited to, lymphomas, myelomas, autoimmune diseases, and transplant rejection.

The methods of the present invention encompass methods of inhibiting and/or killing an activated lymphocyte, and methods of inhibiting the proliferation of a lymphocyte. This is because, as demonstrated by the data disclosed herein, the methods of the invention cause a dose and time dependent inhibition of proliferating lymphocytes, as well as dose and time dependent apoptosis in lymphocytes. The methods of the present invention further comprise methods of treating a patient suffering from a disease associated with an activated lymphocyte. Such diseases are known in the art and are disclosed elsewhere herein. The methods of the invention are based, in part, on the novel finding that 5-HT receptor antagonists, such as those disclosed herein, are useful in inhibiting and/or killing activated lymphocytes.

### I. Compositions

The present invention comprises compositions for inhibiting and/or killing activated lymphocytes, for inhibiting proliferation in lymphocytes, and for treating diseases associated with such lymphocytes. One embodiment of the present invention, includes compositions which induce cell death and apoptosis in various activated lymphocytes, including T cells and B cells. The compositions of the present invention include a composition of Formula I, as well as ICI-955, ICI-956, ICI-957, ICI-1247, ICI-1259, ICI-1260, ICI-1451, or ICI-1505. Specific examples of a compound according to formula I include ICI-1506, ICI-1520, ICI-1551, and ICI-1552.

5-HT receptor antagonists having the structure of formula I or ICI-955, ICI-956, ICI-957, ICI-1247, ICI-1259, ICI-1260, ICI-1451, or ICI-1505, are useful in the present invention for inhibiting the proliferation of lymphocytes, such as T cells and B cells, and for inducing apoptosis and/or cell death in lymphocytes. Thus, the compounds of the present invention is useful for treating, among other things, lymphomas, myelomas, autoimmune diseases, transplant rejection, and the like.

The present invention comprises administering an effective amount of a compound according to formula I, ICI-955, ICI-956, ICI-957, ICI-1247, ICI-1259, ICI-1260, ICI-1451, ICI-1505, or a pharmaceutically acceptable salt thereof. Compounds of formula I have the following chemical structure:

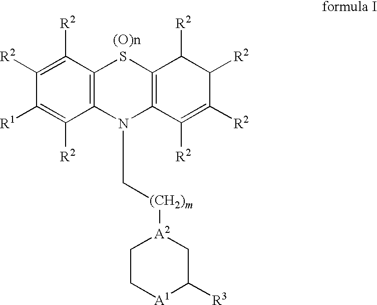

wherein:

R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;

R³ is hydrogen, C(═O)OR⁷, or C(═O)NR⁷₂;

A¹ is NR⁴;

A² is CH or N;

R⁴ is (CH₂)ₚR⁵; C(═O)(CH₂)ₚR⁵; or C(═O)(CH₂)ₚNR⁶₂;

R⁵ is

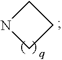

R⁶ is independently selected at each occurrence from the group consisting of hydrogen and (C₁-C₆)alkyl;

R⁷ is independently selected at each occurrence from the group consisting of hydrogen and (C₁-C₆)alkyl;

m is independently at each occurrence 1, 2, or 3;

n is 0, 1, or 2;

p is independently at each occurrence 2 or 3; and

q is independently at each occurrence 1 or 2.

In the definitions of each of the compounds of formula I above:

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tertbutyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable monounsaturated or di-unsaturated straight chain, branched chain or cyclic hydrocarbon group having the stated number of carbon atoms. Examples include vinyl, propenyl (allyl), crotyl, isopentenyl, butadienyl, 1,3-pentadienyl, 1,4-pentadienyl, cyclopentenyl, cyclopentadienyl and the higher homologs and isomers. A functional group representing an alkene is exemplified by CH═CHCH₂.

The term “alkylene”, by itself or as part of another substituent means, unless otherwise stated, a divalent straight, branched or cyclic chain hydrocarbon.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “heteroaryl” refers to a heterocycle having aromatic character. A polycyclic heteroaryl may include one or more rings which are partially saturated. Examples include tetrahydroquinoline and 2,3 dihydrobenzofuryl. For compounds of formula I, the attachment point is understood to be on an atom which is part of an aromatic monocyclic ring or a ring component of a polycyclic aromatic which is itself an aromatic ring.

Examples of heteroaryl groups include: pyridyl, pyrazinyl, pyrimidinyl, particularly 2 and 4 pyrimidinyl, pyridazinyl, thienyl, furyl, pyrrolyl, particularly 2 pyrrolyl, imidazolyl, thiazolyl, oxazolyl, pyrazolyl, particularly 3 and 5 pyrazolyl, isothiazolyl, 1,2,3 triazolyl, 1,2,4 triazolyl, 1,3,4 triazolyl, tetrazolyl, 1,2,3 thiadiazolyl, 1,2,3 oxadiazolyl, 1,3,4 thiadiazolyl and 1,3,4 oxadiazolyl.

Examples of polycyclic heterocycles include: indolyl, particularly 3, 4, 5, 6 and 7 indolyl, indolinyl, quinolyl, tetrahydroquinolyl, isoquinolyl, particularly 1 and 5 isoquinolyl, 1,2,3,4 tetrahydroisoquinolyl, cinnolinyl, quinoxalinyl, particularly 2 and 5 quinoxalinyl, quinazolinyl, phthalazinyl, 1,8 naphthyridinyl, 1,4 benzodioxanyl, coumarin, dihydrocoumarin, benzofuryl, particularly 3,4,1,5 naphthyridinyl, 5, 6 and 7 benzofuryl, 2,3 dihydrobenzofuryl, 1,2 benzisoxazolyl, benzothienyl, particularly 3, 4, 5, 6, and 7 benzothienyl, benzoxazolyl, benzthiazolyl, particularly 2 benzothiazolyl and 5 benzothiazolyl, purinyl, benzimidazolyl, particularly 2 benzimidazolyl, benztriazolyl, thioxanthinyl, carbazolyl, carbolinyl, acridinyl, pyrrolizidinyl, and quinolizidinyl.

The aforementioned listing of heteroaryl moieties is intended to be representative and not limiting.

The term halogen means, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —CF₃.

The compounds of formula I, ICI-955, ICI-956, ICI-957, ICI-1247, ICI-1259, ICI-1260, ICI-1451, and ICI-1505 can be prepared by a person skilled in the art of synthetic organic chemistry. The person skilled in the art knows how to select and implement appropriate synthetic routes. Suitable synthetic methods may be identified by reference to the literature describing synthesis of analogous compounds, and then performing the synthesis of the desired compound following the route used for the analogous compounds, modifying the starting materials, reagents, and reaction conditions as appropriate to synthesizing any particular desired compounds. In addition, reference may be made to sources such as Comprehensive Organic Synthesis, Ed. B. M. Trost and I. Fleming (Pergamon Press 1991), Comprehensive Organic Functional Group Transformations, Ed. A. R. Katritzky, O. Meth Cohn, and C. W. Rees (Pergamon Press, 1996), Comprehensive Organic Functional Group Transformations II, Ed. A. R. Katritzky and R. J. K. Taylor (Editor) (Elsevier, 2nd Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996), the entire disclosures of which are incorporated herein by reference.

It will be understood that when compounds of formula I, ICI-955, ICI-956, ICI-957, ICI-1247, ICI-1259, ICI-1260, ICI-1451, or ICI-1505 contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are efficacious in the treatment of diseases associated with activated and/or proliferating lymphocytes, including, but not limited to, lymphomas, myelomas, autoimmune diseases, and transplant rejection.

The isomers resulting from the presence of a chiral center comprise a pair of non superimposable isomers that are called “enantiomers.” Single enantiomers of a pure compound are optically active, i.e., they are capable of rotating the plane of plane polarized light.

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

By “isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well known chiral separation techniques. According to one such method, a racemic mixture of a compound having the structure of Formula I, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

The present invention further comprises compositions for inhibiting and/or killing activated lymphocytes, for inhibiting proliferating lymphocytes, and for treating diseases associated with such lymphocytes. One embodiment of the present invention includes compositions which, as demonstrated by the data disclosed herein, induce cell death and apoptosis in a variety of activated lymphocytes, including T cells and B cells.

The compositions of the present invention include, but are not limited to, the compositions disclosed below.

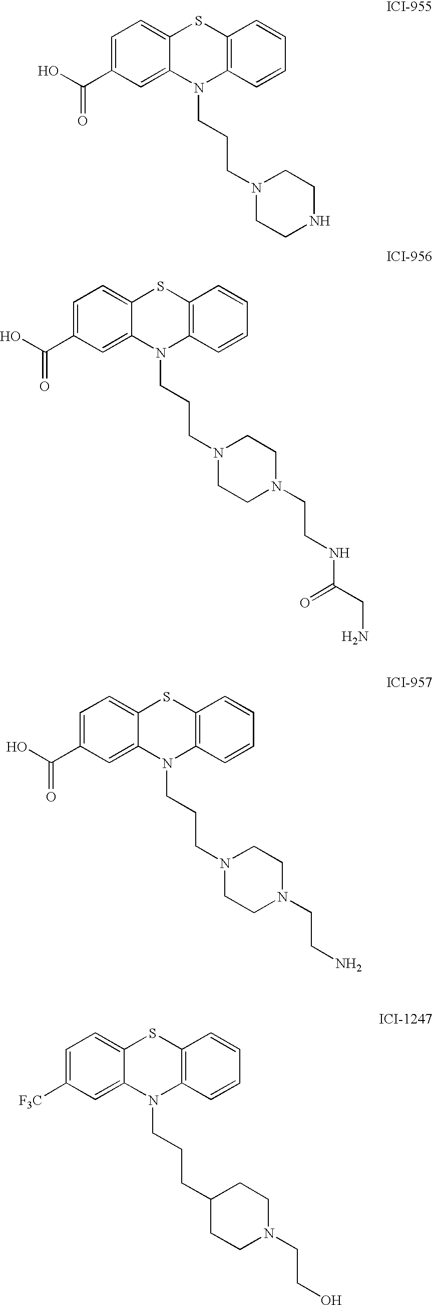

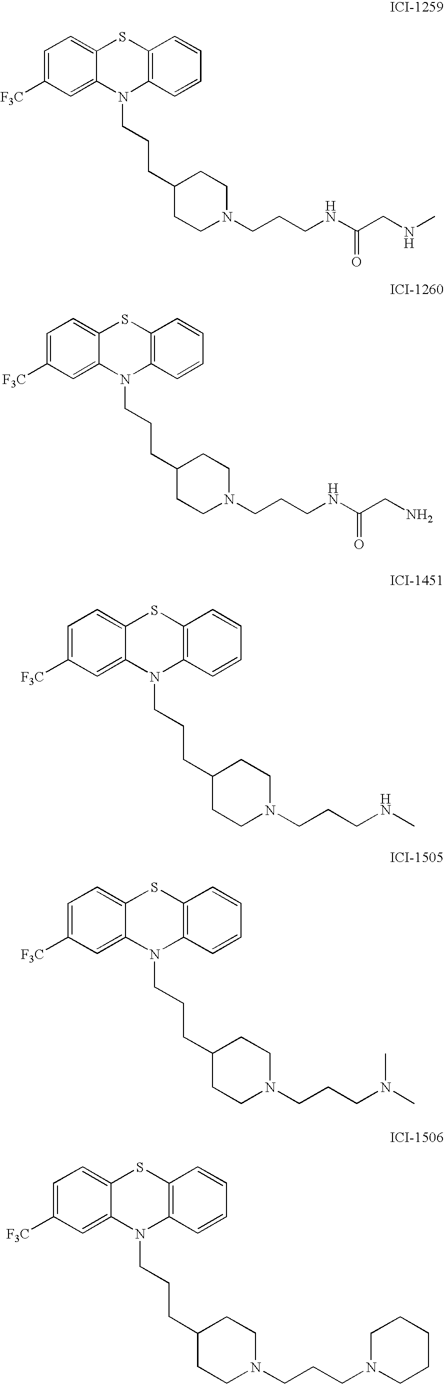

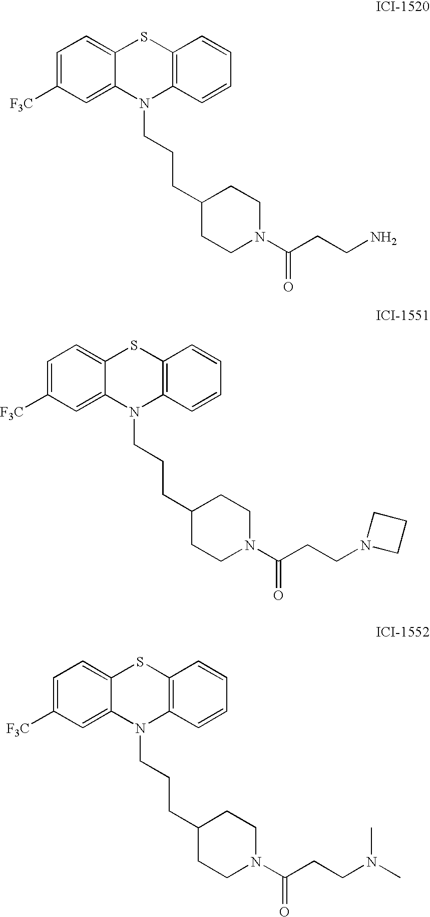

The compounds of the present invention can be used or administered as a pharmaceutically acceptable salt. The phrase “pharmaceutically acceptable salt(s)”, as used herein, unless otherwise indicated, includes salts of acidic or basic groups which may be present in the compounds disclosed herein. The compounds disclosed herein that are basic in nature are capable of forming a wide variety of salts with various inorganic and organic acids. The acids that may be used to prepare pharmaceutically acceptable acid addition salts of such basic compounds of the present 5-HT receptor antagonists are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, dislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Since a single compound of the present invention may include more than one acidic or basic moieties, the compounds of the present invention may include mono, di or tri-salts in a single compound.

The 5-HT receptor antagonists of the present invention that are acidic in nature are capable of forming base salts with various pharmacologically acceptable cations. Examples of such salts include the alkali metal or alkaline earth metal salts and, particularly, the calcium, magnesium, sodium and potassium salts of the compounds of the present invention.

This invention also encompasses pharmaceutical compositions comprising prodrugs of the present 5-HT receptor antagonists. Compounds of formula I as well as ICI-955, ICI-956, ICI-957, ICI-1247, ICI-1259, ICI-1260, ICI-1451, ICI-1505, and the other 5-HT receptor antagonists disclosed herein having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds disclosed herein. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19: 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Free amines can also be derivatized as amides, sulfonamides or phosphonamides. All of these prodrug moieties may incorporate groups including but not limited to ether, amine and carboxylic acid functionalities.

The present invention also includes isotopically-labeled compounds, which are identical to those recited in the 5-HT receptor antagonists of the invention, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, 31P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., ³H, and ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed herein and known in the art by substituting a readily available isotopically labeled reagent for a non-isotopically labeled reagent.

The compounds of the present invention can also be combined with other compounds useful in the treatment of diseases such as autoimmune diseases, lymphomas, myelomas, and transplant rejection. Such compounds include, but are not limited to, the following therapeutic agents: dexamethasone, melphalan, doxorubicin, bortezomib, lenalidomide, thalidomide, and other agents, such as, but not limited to, regulators of gene expression (e.g., steroids and glucocorticoids, alkylating agents that are known mutagens (e.g., cyclophosphamide), inhibitors of kinases and phosphatases which act on the calcineurin and JNK/p38 kinase pathways and the cyclin kinase cascade (e.g., CyclosporinA, Tacrolimus [FK506], and Rapamycin), inhibitors of de novo purine synthesis which act as inhibitors of guanosine nucleotide synthesis and are used to prevent allograft rejection and to treat ongoing rejection (e.g., Mycophenolate motefil), and inhibitors of de novo pyrimidine synthesis which are used to treat patients afflicted with rheumatoid arthritis (e.g., Leflunomide), TNF-α inhibitors, such as Adalimumab, Etanercept, Infliximab, and other immunomodulating agents, such as methotrexate, azathioprine, natalizumab, and mercaptopurine. Therefore, the invention encompasses a composition comprising a 5-HT receptor antagonist disclosed herein, such as a 5-HT receptor antagonist of formula I, and immunomodulating agent disclosed elsewhere herein.

A composition comprising a compound of the present invention, such as the 5-HT receptor antagonist of formula I, ICI-955, ICI-956, ICI-957, ICI-1247, ICI-1259, ICI-1260, ICI-1451, ICI-1505, or another compound disclosed herein, and a therapeutic agent are within the scope of the present invention, whether physically combined prior to administration to a patient or combined within a patient.

### II. Methods

A. Methods of Inducing Apoptosis and Inhibiting Proliferation in a Lymphocyte

The present invention includes a method of inducing apoptosis in a lymphocyte. The method comprises inhibiting the interaction of serotonin with a serotonin receptor by contacting a lymphocyte with a 5-HT receptor antagonist, such as a compound of formula I, ICI-955, ICI-956, ICI-957, ICI-1247, ICI-1259, ICI-1260, ICI-1451, or ICI-1505.

Contacting a lymphocyte with a 5-HT receptor antagonist of the present invention results in, among other things, an inhibition of proliferation of a variety of lymphocytes, including T-cells and B-cells. In addition, contacting a lymphocyte with a 5-HT receptor antagonist of the present application results in apoptosis of the lymphocyte in a dose and time dependent manor. Thus, the present invention comprises inducing apoptosis in a lymphocyte and a method of inhibiting proliferation of a lymphocyte by contacting the lymphocyte with a 5-HT receptor antagonist.

The present invention further comprises a method of treating a mammal, preferably a human, having a disease characterized by abnormal lymphocyte proliferation where inhibiting lymphocyte proliferation or inducing apoptosis in the abnormally proliferating lymphocytes results in treatment of the disease. The method comprises administering an effective amount of a 5-HT receptor antagonist to a mammal, preferably a human, in need thereof. Administration of a 5-HT receptor antagonist of the present invention results in, among other things, a rapid cessation of proliferation of various types of lymphocytes, including, but not limited to, T-cells and B-cells. In addition, according to the data presented herein, administration of a 5-HT receptor antagonist of the present invention results in apoptosis in the lymphocyte. Inducing apoptosis or inhibiting proliferation of a lymphocyte prevents or treats the generation of an immune response, such as those common to autoimmune diseases and transplant rejection, and also treats lymphatic neoplasias, including lymphomas and myelomas.

One of skill in the art would appreciate, based upon the disclosure provided herein, that the invention encompasses using a 5-HT receptor antagonist that is water soluble and that does not substantially cross the blood-brain barrier. Blood-brain barrier impermeability is important in order to selectively inhibit signaling via serotonin receptor on an immune cell while not affecting serotonin signaling via a serotonin receptor on a neural cell. Accordingly, the present invention encompasses using a compound that while inhibiting serotonin signaling via a serotonin receptor on a cell, does not substantially cross the blood-brain barrier. Such compounds are disclosed elsewhere herein and include the 5-HT receptor antagonist of formula I, ICI-955, ICI-956, ICI-957, ICI-1247, ICI-1259, ICI-1260, ICI-1451, and ICI-1505. The art is repelet with assays for assessing the ability of a substance to cross the barrier.

The compounds of the present invention can be used to treat a variety of autoimmune diseases, including, but not limited to, myasthenia gravis, idiopathic inflammatory myopathy, chronic neutropenia, rheumatoid arthritis, idiopathic thromcytopenia purpura, autoimmune hemolytic syndromes, antiphospholipid antibody syndromes, inflammatory bowel disease, Crohn's disease, ulcerative colitis, myocarditis, Guillian-Barre Syndrome, vasculitis, multiple sclerosis, neuromyelitis optica (devic's syndrome), lymphocytic hypophysitis, Graves disease, Addison's disease, hypoparathroidism, type 1 diabetes, systemic lupus erythematosus, pemphigus vulgaris, bullous pemphigoid, psoriasis, psoriatic arthritis, endometriosis, autoimmune orchitis, dystrophic epidermolysis, sarcoidosis, Wegener's granulomatosis, autoimmune deafness, Sjögren's disease, autoimmune uveoretinitis, interstitial cystitis, Goodpasture's syndrome, and fibromyalgia.

The ability of the compounds of the invention to treat the above recited diseases is due to the fact that the 5-HT receptor antagonists of the present invention inhibit the proliferation of both T cells and B cells, and additionally induce apoptosis in such lymphocytes. Thus, the methods of the present invention comprise administering an effective amount of a 5-HT receptor antagonist to a mammal, preferably a human, having an autoimmune disease, e.g. psoriasis.

The invention further comprises compounds and methods for treating asthma.

The present invention also comprises compositions and methods for the treatment of immune-cell related diseases and disorders. In an aspect, the disease or disorder is not autoimmune-related.

The present invention further comprises a method of treating organ transplant rejection in a mammal in need thereof. Specifically contemplated in the present invention are methods of treating graft versus host disease (GVHD) and organ transplant rejection by administering a 5-HT receptor antagonist disclosed herein to a patient suffering from GVHD and/or organ transplant rejection. The present invention comprises methods of treating, for example, transplant rejection of thoracic organs, such as heart transplants, lung transplants and en bloc heart/lung transplants. The methods of the invention further comprise treating rejection of abdominal organs, such as liver, kidney, pancreas, small bowel and combined transplants, such as kidney/pancreas transplants, liver/kidney transplants, and combined liver/small bowel transplants. The methods of the present invention further comprise treatment after rejection of a hand, cornea, skin or face transplant. In addition, the methods of the present invention can be used to treat rejection of tissues, cells and fluids that are commonly transplanted, including, but not limited to, pancreatic islet cells (islets of Langerhans), bone marrow transplants, adult stem cell transplants, blood transfusions, blood vessel grafts, heart valve grafts, where autologous, allogenic or xenogenic, and bone grafts.

The ability of the compounds of the invention to treat GVHD is due to the fact that the 5-HT receptor antagonists of the present invention inhibit proliferation of T cells, one of the effector cells in transplant and graft rejection, and induce apoptosis in B cells, which produce anti-graft antibodies. Thus, the invention encompasses a method of treating transplant rejection by administering an effective amount of the 5-HT receptor antagonists of the present invention to a mammal, preferably a human, in need thereof.

The methods of the present invention further comprise treating a mammal having an autoimmune disease or a mammal rejecting an organ or tissue transplant with a combination of a 5-HT receptor antagonist with another immunomodulatory agent. Such immunomodulatory agents include, but are not limited to, other agents, such as, but not limited to, regulators of gene expression (e.g., steroids and glucocorticoids, alkylating agents that are known mutagens (e.g., cyclophosphamide), inhibitors of kinases and phosphatases which act on the calcineurin and JNK/p38 kinase pathways and the cyclin kinase cascade (e.g., CyclosporinA, Tacrolimus [FK506], and Rapamycin), inhibitors of de novo purine synthesis which act as inhibitors of guanosine nucleotide synthesis and are used to prevent allograft rejection and to treat ongoing rejection (e.g., Mycophenolate motefil), and inhibitors of de novo pyrimidine synthesis which are used to treat patients afflicted with rheumatoid arthritis (e.g., Leflunomide), TNF-α inhibitors, such as Adalimumab, Etanercept, Infliximab, and other immunomodulating agents, such as methotrexate, azathioprine, natalizumab, and mercaptopurine.

The immunomodulatory agents of the present invention can be combined with a 5-HT receptor antagonist of the present invention, such as the 5-HT receptor antagonist of formula I, ICI-955, ICI-956, ICI-957, ICI-1247, ICI-1259, ICI-1260, ICI-1451, or ICI-1505 to treat a patient having an autoimmune disease or a patient experiencing transplant rejection. The immunomodulatory agent can be combined with a 5-HT receptor antagonist and delivered as one dose or a series of doses, either together or separately. Methods for the combinations of drugs and dosages are described elsewhere herein.

The present invention further comprises a method of treating neoplasias in a human, preferably lymphomas and myelomas. Neoplastic lymphoma and myeloma cells, when contacted with a 5-HT receptor antagonist of the present invention, cease proliferating and apoptose. Thus, the present invention comprises methods for treating a mammal, preferably a human, having a lymphoma or a myeloma, the method comprising administering to the mammal an effective amount of a 5-HT receptor antagonist of the present invention. Such 5-HT receptor antagonists include, but are not limited to the 5-HT receptor antagonist of formula I, as well as ICI-955, ICI-956, ICI-957, ICI-1247, ICI-1259, ICI-1260, ICI-1451, and ICI-1505.

A mammal having a lymphoma can be treated using the methods of the present invention by administering to the mammal an effective amount of a 5-HT receptor antagonist of the present invention. Lymphomas that can be treated using the methods of the present invention include, but are not limited to, non-Hodgkin lymphomas, such as T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, and anaplastic large cell lymphoma. The present invention further comprises methods of treating Hodgkin's lymphomas by administering to a patient having a Hodgkin's lymphoma an effective amount of a 5-HT receptor antagonist of the present invention. Such Hodgkin's lymphomas include, but are not limited to, nodular lymphocyte-predominant Hodgkin lymphoma and classical Hodgkin lymphoma, including nodular sclerosis, mixed cellularity Hodgkin's lymphoma, lymphocyte-rich Hodgkin's lymphoma and lymphocyte depleted Hodgkin's lymphoma.

The methods of the present invention further comprise treating a mammal, preferably a human, with myeloma. The 5-HT receptor antagonists of the present invention inhibit the proliferation and induce apoptosis in a variety of common myeloma cells, including primary multiple myeloma cells from treated and untreated patients, and multiple myeloma cells resistant to conventional multiple myeloma therapeutics, such as dexamethasone and melphalan.

The methods of the present invention may be used to treat multiple myeloma in a patient in need thereof. The method comprises administering to a patient in need thereof a compound of the present invention. This method is effective as contacting a multiple myeloma cell with a 5-HT receptor antagonist of the present invention, such as the 5-HT receptor antagonist of formula I, ICI-955, ICI-956, ICI-957, ICI-1247, ICI-1259, ICI-1260, ICI-1451, or ICI-1505 inhibits proliferation of the multiple myeloma cell as well as induce apoptosis in a multiple myeloma cell. Thus, the present invention comprises a method of treating multiple myeloma in a mammal, preferably a human. Further, the present invention comprises a method of inducing apoptosis in a multiple myeloma cell, whether in a patient or isolated from the patient, by contacting the multiple myeloma cell with a compound of the present invention.

The present invention is used to treat multiple myeloma of all stages on the International Staging System (ISS), including Stage I: P2-microglobulin <3.5 mg/L, albumin ≧3.5 g/dL; Stage II: P2-microglobulin <3.5 mg/L and albumin <3.5 g/dL or P2-microglobulin between 3.5 and 5.5 mg/L; and Stage III: β2-microglobulin >5.5 mg/L. In addition, the methods of the present invention comprise combination therapy for treating multiple myeloma. The combinations of the present invention comprise a 5-HT receptor antagonist, such as the 5-HT receptor antagonist of formula I or ICI-955, ICI-956, ICI-957, ICI-1247, ICI-1259, ICI-1260, ICI-1451, and ICI-1505 combined with an additional agents and therapies used for treating multiple myeloma. Specifically contemplated combination therapies include a 5-HT receptor antagonist administered before or after allogeneic or autologous stem cell transplantation, a 5-HT receptor antagonist and a bisphosphonate (e.g. pamidronate) to prevent fractures, and a 5-HT receptor antagonist and erythropoietin to treat anemia associated with multiple myeloma.

Additional combination therapies specifically contemplated in the present invention include a 5-HT receptor antagonist and dexamethasone with or without thalidomide, a 5-HT receptor antagonist and thalidomide, a 5-HT receptor antagonist and vincristine, a 5-HT receptor antagonist and doxorubicin, a 5-HT receptor antagonist and melphalan, and a 5-HT receptor antagonist with melphalan and prednisone. In relapsed patients, or patients otherwise not responding to conventional multiple myeloma therapies, the invention encompasses methods of treating multiple myeloma in a patient comprising administering combinations of a 5-HT receptor antagonist and cyclophosphamide, a 5-HT receptor antagonist and bortezomib or a 5-HT receptor antagonist and lenalidomide. The renal failure that often accompanies multiple myeloma can be treated using a 5-HT receptor antagonist of the present invention and kidney dialysis.

The combinations of a 5-HT receptor antagonist and another multiple myeloma therapy is effective at inhibiting proliferation and inducing apoptosis in multiple myeloma cells. The 5-HT receptor antagonists of the present invention likewise induce apoptosis and inhibit proliferation in a variety of lymphocytes, and thus are useful in the treatment of various immune system related diseases. Thus, the present invention further comprises a method of inhibiting an immune response in a mammal, preferably a human. Inhibition is accomplished by inhibiting serotonin binding with a serotonin receptor through administering a 5-HT receptor antagonist of the present invention. The compound of the invention inhibit an immune reaction in the cell, which in turn inhibits an immune response mediated by that cell.

The invention further comprises a method of inhibiting an immune reaction by an immune cell. This is because, as set forth elsewhere herein, inhibition of serotonin binding with a serotonin receptor on the immune cell inhibits activation of the cell, which in turn inhibits an immune reaction by that cell when compared to the immune reaction by that cell in the absence of inhibition of serotonin binding and/or when compared with the immune reaction of an otherwise identical cell wherein serotonin binding with its receptor is not inhibited.

The present invention further encompasses a method of inhibiting activation of an immune cell, such as a lymphocyte, in a mammal, preferably, a human, wherein the activation is mediated by activation of a serotonin receptor on the cell. Again, this is because, inhibiting serotonin signaling via a serotonin receptor on an immune cell by contacting the cell with a 5-HT receptor antagonist inhibits activation of the cell, and therefore, also inhibits the immune response that would otherwise be produced by that cell.

The 5-HT receptor antagonist, alone or in combinations described herein, that inhibits the serotonin receptor-mediated signals can be administered to a cell, a tissue, or an animal to inhibit interaction of serotonin with a serotonin type receptor on a cell, a tissue, or in an animal. Methods for the safe and effective administration of the 5-HT receptor antagonists described herein are know to those skilled in the art. For instance, the administration of serotonin antagonists is described in the standard literature. That is, the administration of many serotonin-affecting agents, serotonin receptor antagonists, and fluphenazine is set forth in the Physician's Desk Reference (1996 edition, Medical Economics Co., Montvale, N.J.), the disclosure of which is incorporated by reference as if set forth in its entirety herein.

For administration of a 5-HT receptor antagonist of the present invention to a mammal, the compound can be suspended in any pharmaceutically acceptable carrier, for example, sterile water or a buffered aqueous carriers, such as glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey), the disclosure of which is incorporated by reference as if set forth in its entirety herein.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides.

Pharmaceutical compositions that are useful in the methods of the invention may be administered, prepared, packaged, and/or sold in formulations suitable for oral, rectal, vaginal, parenteral, topical, pulmonary, intranasal, buccal, ophthalmic, bolus injection, or another route of administration. Other contemplated formulations include projected nanoparticles, liposomal preparations, resealed erythrocytes containing the active ingredient, and immunologically-based formulations.

The compositions of the invention may be administered via numerous routes, including, but not limited to, oral, rectal, vaginal, parenteral, topical, pulmonary, intranasal, buccal, or ophthalmic administration routes. The route(s) of administration will be readily apparent to the skilled artisan and will depend upon any number of factors including the type and severity of the disease being treated, the type and age of the veterinary or human patient being treated, and the like.

Pharmaceutical compositions that are useful in the methods of the invention may be administered systemically in oral solid formulations, ophthalmic, suppository, aerosol, topical or other similar formulations. In addition to the compound such as heparan sulfate, or a biological equivalent thereof, such pharmaceutical compositions may contain pharmaceutically-acceptable carriers and other ingredients known to enhance and facilitate drug administration.

Compounds which are identified using any of the methods described herein may be formulated and administered to a mammal for treatment of immune system conditions (i.e., autoimmune diseases and allograft rejection), are now described.

The invention encompasses the preparation and use of pharmaceutical compositions comprising a compound useful for treatment of a wide variety of disorders such as T cell lymphomas, autoimmune disorders (see infra), complications arising from solid organ transplants, skin graft rejection, graft versus host disease in bone marrow transplants, multiple myeloma, and the like.

The pharmaceutical compositions described herein can be prepared alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, subcutaneous, intraperitoneal, intramuscular, intrasternal injection, bolus injections, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 0.1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e., by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed. (1985, Remington's Pharmaceutical Sciences, Mack Publishing Co., Easton, Pa.), which is incorporated herein by reference.

Typically, dosages of the compound of the invention which may be administered to an animal, preferably a human, will vary depending upon any number of factors, including but not limited to, the type of animal and type of disease state being treated, the age of the animal and the route of administration. Appropriate dosages may include, but are not limited, doses in the range from about 0.01 mg/kg to about 100 mg/kg, as well as any whole or partial increment therebetween. Preferred dosages include 1 mg/kg and 0.1 mg/kg, administered orally and i.p., respectively.

The compound can be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, and the like. Preferably, the compound is, but need not be, administered as a bolus injection that provides lasting effects for at least one day following injection. The bolus injection can be provided intraperitoneally.

Thus, the skilled artisan would appreciate, once armed with the teachings provided herein, that the invention encompasses administration of a bolus comprising an inhibitor of the interaction of serotonin with a serotonin receptor. Preferably, the inhibitor is a 5-HT receptor antagonist of formula I or ICI-955, ICI-956, ICI-957, ICI-1247, ICI-1259, ICI-1260, ICI-1451, or ICI-1505. Without wishing to be bound by any particular theory, administration of a bolus dose mediates apoptosis of certain cells, such as, among others, an activated T cell or a cancerous B cell (such as, e.g., a multiple myeloma cell), such that repeated doses of the inhibitor is not necessary since the bolus mediates the death of memory, or other, cells that would otherwise mediate the immune response that would otherwise cause the transplanted cell or tissue to be rejected. This effect can be mediated by a localized concentration of a 5-HT receptor antagonist at the 5HTR1B receptor, which concentration is sufficient to inhibit transmission of the serotonin signal, thereby mediating cell death and/or inhibition of an immune response by the cell.

### III. Kits

The invention encompasses various kits relating to inhibiting the interaction of serotonin with a serotonin receptor because, as disclosed elsewhere herein, inhibiting this interaction in turn inhibits activation of an immune cell thereby inhibiting an immune response. Thus, in one aspect, the invention includes a kit for modulating an immune response in a mammal. The kit comprises an effective amount of an inhibitor of the interaction of serotonin with a serotonin receptor. Such an inhibitor includes, preferably, a serotonin receptor antagonist. And the kit further comprises an applicator and an instructional material for the use thereof.

Additionally, one skilled in the art would appreciate, based upon the disclosure provided herein, that the inhibitor can be a compound that does not cross the blood-brain barrier and is preferably water soluble. This is because, as more fully discussed elsewhere herein, it may be desirable to inhibit serotonin signaling in a non-neural cell, while not affecting such signaling in a neural cell, which would be protected beyond the blood-brain barrier.

In a specific embodiment, the kit of the present invention comprises a 5-HT receptor antagonist, an applicator, and an instructional material for the use thereof. In another embodiment, the kit can comprise a 5-HT receptor antagonist, such as those described elsewhere herein, a container holding the 5-HT receptor antagonist, and an instructional material. The skilled artisan can provide the applicator.

Preferably, the kit of the present invention comprises a 5-HT receptor antagonist of formula I or ICI-955, ICI-956, ICI-957, ICI-1247, ICI-1259, ICI-1260, ICI-1451, or ICI-1505. Additionally, the kit can comprise an instructional material and an applicator for the administration of a 5-HT receptor antagonist of the present invention.

The kits of the present invention can be used to treat the diseases and conditions disclosed elsewhere herein. Specifically, the kits of the present invention can be used to treat, among other things, autoimmune diseases, such as psoriasis, organ transplant rejection, such as kidney transplant rejection, lymphoma, such as Hodgkin's lymphoma or non-Hodgkin's lymphoma, and B-cell neoplasias, such as multiple myeloma. The kits described in the present invention are not limited to the uses above however, and can be used in any method derived from the teachings disclosed herein.

### EXAMPLES

The invention is now described with reference to the following synthetic Examples and biological studies. The synthetic Examples disclosed herein are provided for the purpose of illustration only and the invention should in no way be construed as being limited to compounds produced by these synthetic routes, but rather should be construed to encompass any and all variations of these routes which become evident or would be generally apparent to one of ordinary skill in the art as a result of the teaching provided herein.

Similarly, the biological studies described herein are provided for the sole purpose of illustrating the efficacy and/or activity of the compounds disclosed herein. These biological studies should not, however, be construed as limiting the utility or efficacy of these compounds to the animals or systems described below, but instead should be construed to teach the utility of these compounds in all animals and systems that would be generally apparent to one of ordinary skill in the art.

### Starting Materials

Starting material ICI-847, 3-(4-(3-(2-(trifluoromethyl)-10H-phenothiazin-10-yl)propyl)piperidin-1-yl)propan-1-amine, was prepared as disclosed in U.S. patent application Ser. No. 11/897,598.

Starting material ICI-824, 10-(3-(piperidin-4-yl)propyl)-2-(trifluoromethyl)-10H-phenothiazine, was prepared as disclosed in U.S. patent application Ser. No. 11/897,598.

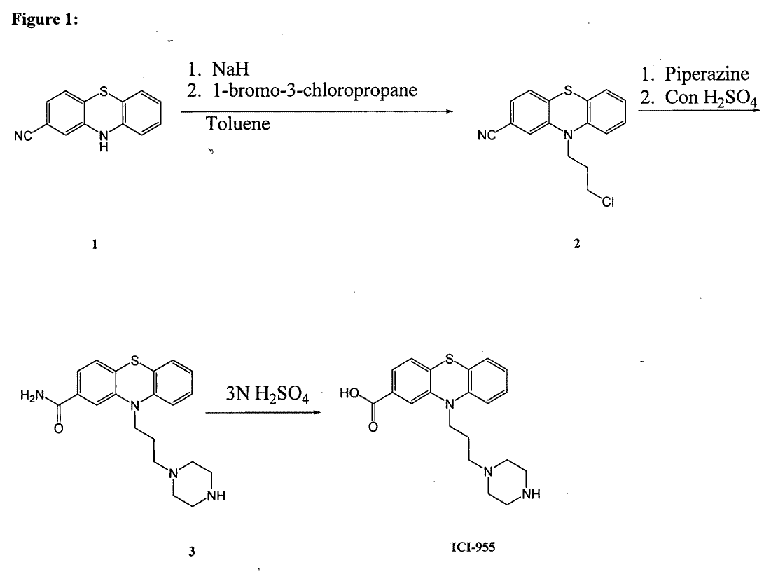

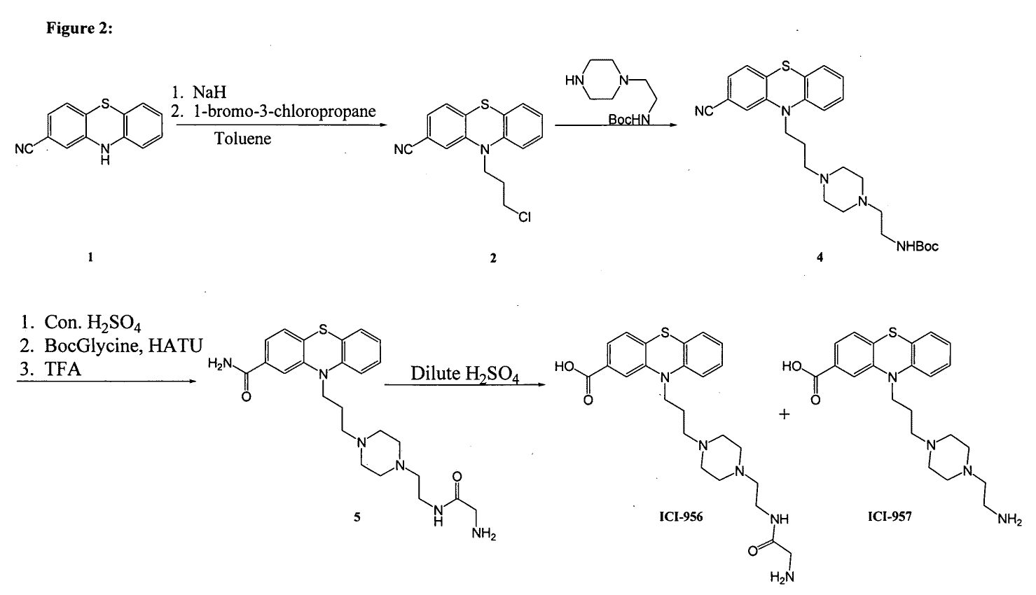

Compounds 2, 3, 4, and 5 were prepared as set forth in FIGS. 1 and 2. Compounds 2, 3, and 4 in these figures were prepared in an analogous fashion to compounds 2 and 3 as set forth in U.S. patent application Ser. No. 11/897,598.

All compounds were purified by reversed-phase HPLC, unless otherwise stated. ¹H NMR spectra and MS data was obtained for all compounds and intermediates.

### Preparative Procedures:

10-(3-(piperazin-1-yl)propyl)-10H-phenothiazine-2-carboxylic acid (“ICI-955”): Compound 3 (FIG. 1) (15 mg, 0.040 mmol) was dissolved in 2.0 mL of 3N H₂SO₄. The solution was stirred at 60° C. overnight. The reaction mixture was evaporated and the resulting residue was purified by reversed-phase HPLC to give pure ICI-955 (9.0 mg, 60%,) as a white solid after lyophilization.
10-(3-(4-(2-(2-aminoacetamido)ethyl)piperazin-1-yl)propyl)-10H-phenothiazine-2-carboxylic acid (“ICI-956”); and 10-(3-(4-(2-aminoethyl)piperazin-1-yl)propyl)-10H-phenothiazine-2-carboxylic acid, ICI-957: Compound 5 (FIG. 2) (15 mg, 0.032 mmol) was dissolved in 2.0 mL of 3N H₂SO₄. The solution was stirred at 60° C. overnight producing a mixture of ICI-956 and ICI-957. The reaction mixture was evaporated and the resulting residue was purified by reversed-phase HPLC to give pure products ICI-956 (6.0 mg) and ICI-957 (6.0 mg) as white solids after lyophilization.

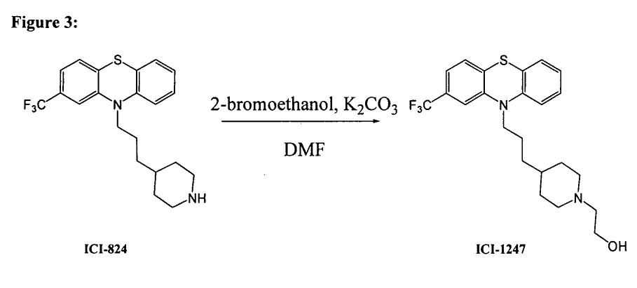

3-(4-(3-(2-(trifluoromethyl)-10H-phenothiazin-10-yl)propyl)piperidin-1-yl)propan-1-ol, (“ICI-1247”): To a solution of ICI-824 (FIG. 3) (50 mg, 0.128 mmol) and K₂CO₃ (200 mg, 1.33 mmol) in dry DMF (5.0 mL) was added 2-bromoethanol (25 mg, 0.20 mmol), and the solution was stirred for 24 h at room temperature. The mixture was diluted with Et₂O and the layers were separated. The aqueous layer was then back-extracted with another portion of Et₂O. The organic layers were then combined, washed with water, brine, dried over Na₂SO₄, filtered, and concentrated in vacuo to give crude ICI-1247. Crude material was purified by reversed phase HPLC to give the desired product as 31.0 mg (70%) after lyophilization.

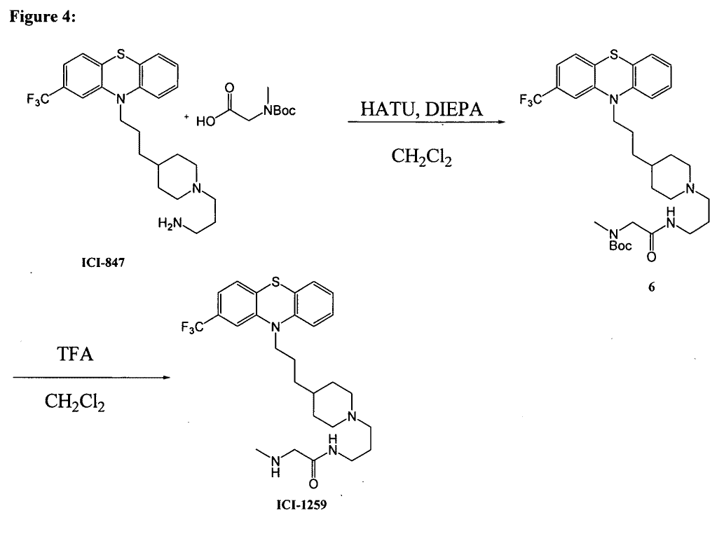

tert-butyl 2-(methylamino)acetyl(3-(4-(3-(2-(trifluoromethyl)-10H-phenothiazin-10-yl)propyl)piperidin-1-yl)propyl)carbamate, (“6”) (FIG. 4): To a solution of boc-sarcosine (32 mg, 0.20 mmol), HATU (77 mg, 0.20 mmol) and ICI-847 (75 mg, 0.17 mmol) in CH₂Cl₂ (15 mL) was added DIPEA (0.1 mL). The mixture was stirred at room temperature for 12 h. The reaction mixture was evaporated to provide crude 6. The residue was purified by a silica gel column chromatography (9:1 CH₂Cl₂:MeOH) to give pure product (63 mg, 60%).

2-(methylamino)-N-(3-(4-(3-(2-(trifluoromethyl)-10H-phenothiazin-10-yl)propyl)piperidin-1-yl)propyl)acetamide, (“ICI-1259”): Compound 6 (63 mg, 0.10 mmol) was dissolved in 5.0 mL of dry CH₂Cl₂ and TFA (0.2 mL, 1.75 mmol) was added dropwise to this solution at 0° C. The solution was stirred at room temperature overnight. The reaction mixture was then evaporated and the resulting residue was purified by reversed-phase HPLC to give 42 mg (80%) of pure ICI-1259.

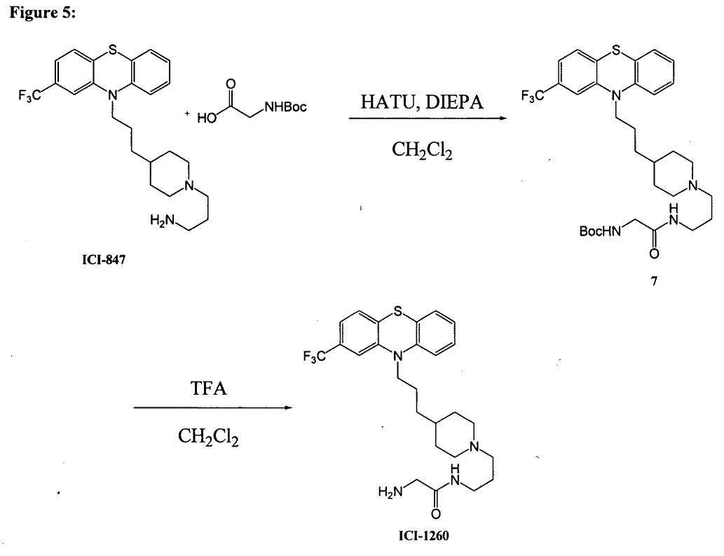

tert-butyl 2-aminoacetyl(3-(4-(3-(2-(trifluoromethyl)-10H-phenothiazin-10-yl)propyl)piperidin-1-yl)propyl)carbamate, (“7”) (FIG. 5): To a solution of boc-glycine (32 mg, 0.20 mmol), HATU (77 mg, 0.20 mmol) and ICI-847 (75 mg, 017 mmol) in CH₂Cl₂ (15 mL) was added DIPEA (0.1 mL) and the mixture was stirred at room temperature for 12 h. The reaction mixture was evaporated and the residue was purified by a silica gel column chromatography (9:1 CH₂Cl₂:MeOH) to give pure, 7 (63 mg, 60%) as a foam.

2-amino-N-(3-(4-(3-(2-(trifluoromethyl)-10H-phenothiazin-10-yl)propyl)piperidin-1-yl)propyl)acetamide, (“ICI-1260”): Compound 7 (63 mg, 0.10 mmol) was dissolved in 5.0 mL of dry CH₂Cl₂ and TFA (0.2 mL, 1.75 mmol) was added dropwise to this solution at 0° C. The solution was stirred at room temperature overnight. The reaction mixture was then evaporated and the resulting residue was purified by reversed-phase HPLC to give 42 mg (80%) of pure ICI-1260.

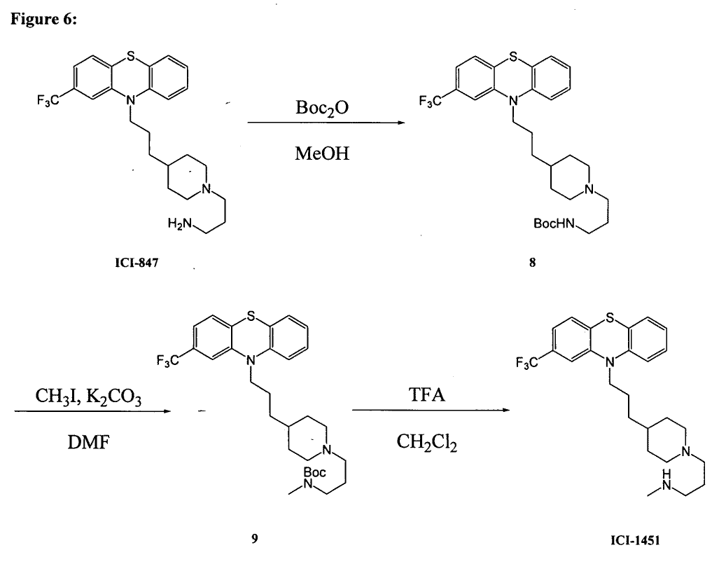

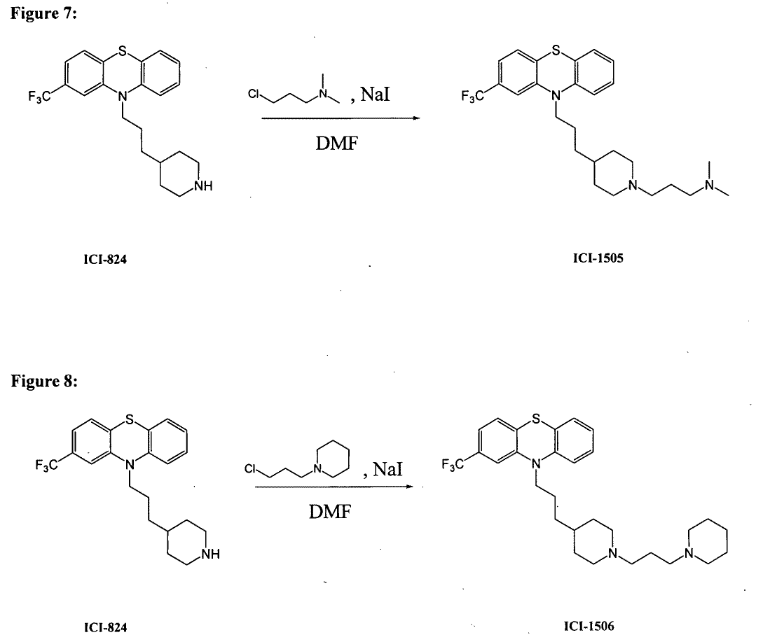

tert-butyl 3-(4-(3-(2-(trifluoromethyl)-10H-phenothiazin-10-yl)propyl)piperidin-1-yl)propylcarbamate, (“8”) (FIG. 6): To a solution of ICI-847 (0.1 g, 0.222 mmol) in MeOH (2.2 mL) was added Boc₂O (0.053 g, 0.244 mmol). The reaction was stirred for 40 minutes before imidazole (8.0 mg, 0.111 mmol) was added. The reaction mixture was stirred for an additional 10 minutes before being concentrated in vacuo. The residue was dissolved in CH₂Cl₂ and the organics washed with 1% HCl in water. The layers were separated and the aqueous layer was extracted with CH₂Cl₂. The organic extracts were combined and dried over Na₂SO₄, filtered and concentrated in vacuo to give crude 8. The crude material was used without further purification.

tert-butyl methyl(3-(4-(3-(2-(trifluoromethyl)-10H-phenothiazin-10-yl)propyl)piperidin-1-yl)propyl)carbamate, (“9”) (FIG. 6): To a mixture of crude 8 and K₂CO₃ (0.037 g, 0.266 mmol) was added DMF (2.3 mL). Subsequently, MeI (15 μL, 0.244 mmol) was added to the reaction mixture and the reaction mixture was heated to 40° C. overnight. The reaction was diluted with EtOAc and washed with water multiple times. The combined aqueous washes were then back-extracted with EtOAc. The combined organic extracts were then dried over Na₂SO₄, filtered and concentrated in vacuo to give crude 9 which was subsequently used without further purification.

N-methyl-3-(4-(3-(2-(trifluoromethyl)-10H-phenothiazin-10-yl)propyl)piperidin-1-yl)propan-1-amine, (“ICI-1451”): Crude 9, was dissolved in CH₂Cl₂ (1.6 mL) and TFA (0.7 mL). The reaction mixture was stirred for 40 minutes, after which it was diluted with water and brought to pH 10 via addition of solid NaHCO₃. The layers were separated and the aqueous layer was extracted with CH₂Cl₂ The combined organic extracts were dried over Na₂SO₄, filtered and concentrated in vacuo. The residue was purified via reversed phase HPLC to give pure ICI-1451.

N,N-dimethyl-3-(4-(3-(2-(trifluoromethyl)-10H-phenothiazin-10-yl)propyl)piperidin-1-yl)propan-1-amine, (“ICI-1505”): To a mixture of ICI-824 (0.05 g, 0.127 mmol), 3-(dimethylamino)propylchloride hydrochloride (0.022 g, 0.139 mmol), K₂CO₃ (0.044 g, 0.318 mmol) and NaI (4.0 mg, 25.4 μmol) was added DMF (1.5 mL). The reaction mixture was warmed to 80° C. and stirred overnight. The reaction mixture was partitioned between water and CH₂Cl₂ and the layers were separated. The aqueous layer was then extracted with fresh CH₂Cl₂. The organic extracts were then combined and washed with water, brine, dried over Na₂SO₄, filtered, and concentrated in vacuo. The residue was purified via reversed phase HPLC to give pure IC-1505.

10-(3-(1-(3-(piperidin-1-yl)propyl)piperidin-4-yl)propyl)-2-(trifluoromethyl)-10H-phenothiazine, (“ICI-1506”): To a mixture of PV-824 (0.05 g, 0.127 mmol), 1-(3-chloropropyl)piperidine monohydrochloride (0.027 g, 0.139 mmol), K₂CO₃ (0.044 g, 0.318 mmol) and NaI (4 mg, 25.4 μmol) was added DMF (1.5 mL). The reaction mixture was warmed to 80° C. and stirred overnight. The reaction mixture was partitioned between water and CH₂Cl₂ and the layers were separated. The aqueous layer was back-extracted with CH₂Cl₂. The organic extracts were then combined, washed with water, brine, dried over Na₂SO₄, filtered, and concentrated in vacuo. The residue was purified via reversed phase HPLC to give pure ICI-1506.

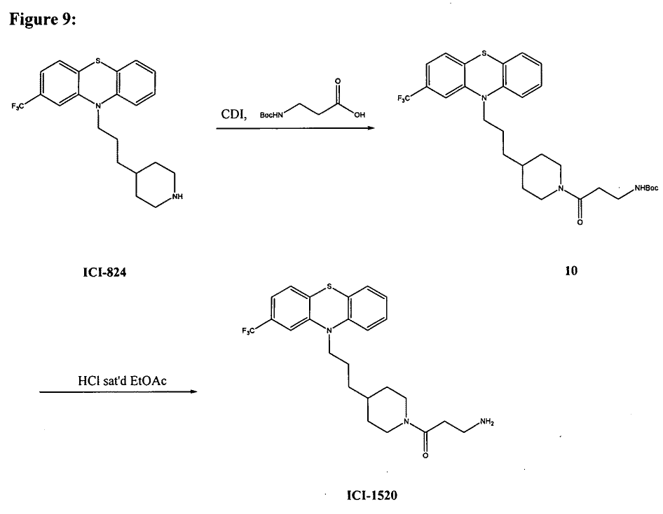

tert-butyl 3-oxo-3-(4-(3-(2-(trifluoromethyl)-10H-phenothiazin-10-yl)propyl)piperidin-1-yl)propylcarbamate, (“10”) (FIG. 9): To a mixture of Boc-β-Ala-OH (0.05 g, 0.264 mmol) and CDI (0.047 g, 0.291 mmol) was added CH₂Cl₂ (2.7 mL). The resulting homogenous solution was stirred for 1.0 hour after which PV-824 (0.114 g, 0.291 mmol) in CH₂Cl₂ (2.0 mL) was added. The reaction mixture was allowed to stir at ambient temperature for 60 hours. The reaction mixture was quenched with saturated aqueous NaHCO₃ and the layers separated. The aqueous layer was then back-extracted with CH₂Cl₂. The organic layers were then combined, dried over Na₂SO₄, filtered, and concentrated in vacuo to give crude 10. The crude material was used without any further purification.

3-amino-1-(4-(3-(2-(trifluoromethyl)-10H-phenothiazin-10-yl)propyl)piperidin-1-yl)propan-1-one, (“ICI-1520”): To a solution of 10 in EtOAc (2.0 mL) was added concentrated HCl (0.7 mL). The resulting reaction mixture was allowed to stir at ambient temperature for 1 hour. Subsequently, the reaction mixture was concentrated in vacuo to give crude IC-1520. The crude product was then purified via reversed phase HPLC.

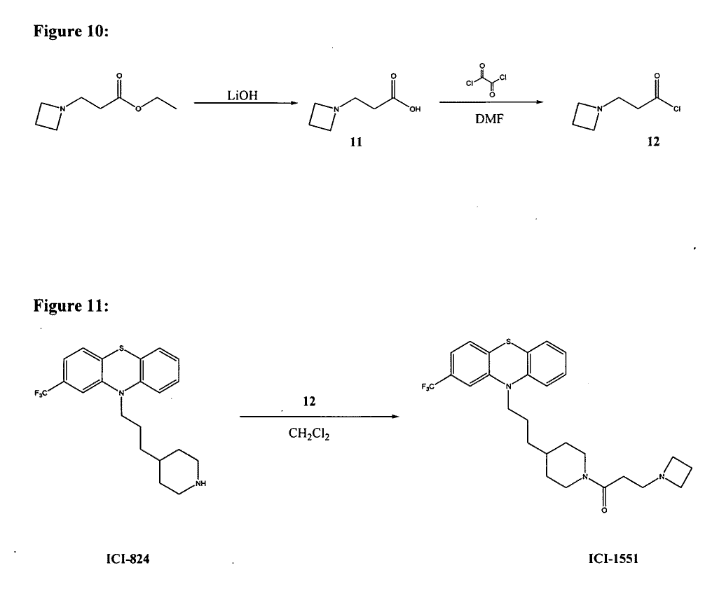

3-(azetidin-1-yl)propanoic acid, (“11”) (FIG. 10): To a solution of ethyl 3-(azetidin-1-yl)propanoate, (0.02 g, 0.127 mmol) in THF (3.8 mL) was added aqueous 1M LiOH (1.9 mL). The reaction mixture was stirred vigorously for 2 hours and then acidified to pH 2 with aqueous 1.0 M HCl. The layers were separated and the aqueous phase was extracted with EtOAc. The organic layers were then combined, dried over Na₂SO₄, filtered, and concentrated in vacuo to give crude 11. The crude acid was used without any further purification.

3-(azetidin-1-yl)propanoyl chloride, (“12”) (FIG. 10): Crude acid 11 was dissolved in oxalyl chloride (2 mL, 2 M in CH₂Cl₂) under an inert atmosphere. Subsequently, DMF (1 drop) was added to the reaction mixture and the resulting solution was stirred at ambient temperature for 1.5 h. The reaction mixture was concentrated in vacuo to give crude acid chloride 12.

3-(azetidin-1-yl)-1-(4-(3-(2-(trifluoromethyl)-10H-phenothiazin-10-yl)propyl)piperidin-1-yl)propan-1-one, (“ICI-1551”): PV-824 (0.055 g, 0.140 mmol) and TEA (0.053 mL, 0.381 mmol) were dissolved in CH₂Cl₂ (1.0 mL) at ambient temperature. Crude acid chloride, 12, (1.0 mL in CH₂Cl₂) was then cannulated into the stirring mixture of ICI-824 and TEA. After stirring at ambient temperature overnight, the reaction mixture was quenched with saturated aqueous NaHCO₃. The layers were then separated and the aqueous layer was extracted with CH₂Cl₂. The organic extracts were then combined, dried over Na₂SO₄, filtered and concentrated in vacuo to give crude ICI-1551. The product was the purified via reversed phase HPLC.

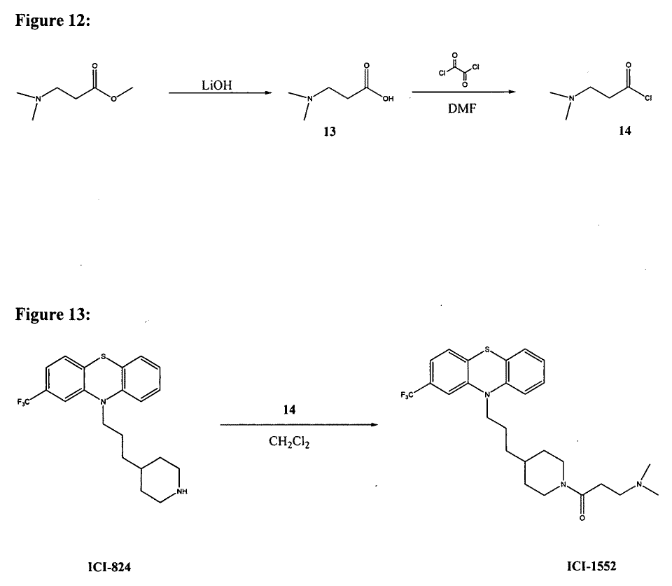

3-(dimethylamino)propanoic acid, (“13”) (FIG. 12): To a solution of methyl 3-(dimethylamino) propionate (0.02 g, 0.152 mmol) in THF (4.6 mL) was added aqueous LiOH (2.3 mL, 1.0 M). The reaction mixture was stirred vigorously for 2 hours and then acidified to pH 2 with aqueous 1.0 M HCl. The layers were separated and the aqueous layer was extracted with EtOAc. The organic layers were then combined, dried over Na₂SO₄, filtered, and concentrated in vacuo to give crude acid. The crude acid was used without any further purification.

3-(dimethylamino)propanoyl chloride, (“14”) (FIG. 12): Crude acid, 13, was dissolved in oxalyl chloride (2.2 mL, 2 M in CH₂Cl₂) under an inert atmosphere. Subsequently, DMF (1 drop) was added to the reaction mixture and the resulting solution was stirred at ambient temperature for 1.5 h. The reaction mixture was concentrated in vacuo to give crude acid chloride, 14.

3-(dimethylamino)-1-(4-(3-(2-(trifluoromethyl)-10H-phenothiazin-10-yl)propyl)piperidin-1-yl)propan-1-one, (“ICI-1552”): PV-824 (0.066 g, 0.167 mmol) and TEA (0.064 mL, 0.456 mmol) were dissolved in CH₂Cl₂ (1.0 mL) at ambient temperature. Crude acyl chloride, 14, (1.0 mL in CH₂Cl₂) was then cannulated into the stirring mixture of PV-824 and TEA. After stirring at ambient temperature overnight, the reaction mixture was quenched with saturated aqueous NaHCO₃. The layers were then separated and the aqueous layer was extracted with CH₂Cl₂. The organic extracts were then combined, dried over Na₂SO₄, filtered and concentrated in vacuo to give crude ICI-1552. The product was the purified via reversed phase HPLC.

### Pharmacokinetic Studies

ICI-1259 and ICI-1260 were supplied as powders and stored frozen until used. Dosing solutions in phosphate buffered saline were prepared at 1 mg/ml. These solutions were then evaluated in fasted male Sprague-Dawley rats. The rats were fitted with a jugular vein cannula for blood sampling. Rats were also fitted with an additional jugular vein cannula, or intradudodenal cannula as needed. Rats were given a commercial rodent diet ad libitum prior to study initiation. Food was then withheld from the animals for a minimum of twelve hours prior to dosing, until four hours postdose when food was returned. Water was supplied ad libitum. Three rats were used in each study, unless otherwise indicated.

For both ICI-1259 and ICI-1260, rats were given a 10 mg/kg dose from a 1 mg/ml solution. The appropriate volume of solution was given as a single bolus dose at time zero on the day of dosing. Blood samples were taken at predose, 5 minutes after dosing, 10 minutes after dosing, 15 minutes after dosing, 30 minutes after dosing, 1 hour after dosing, 2 hours after dosing, 4 hours after dosing, 6 hours after dosing, and 8 hours after dosing.

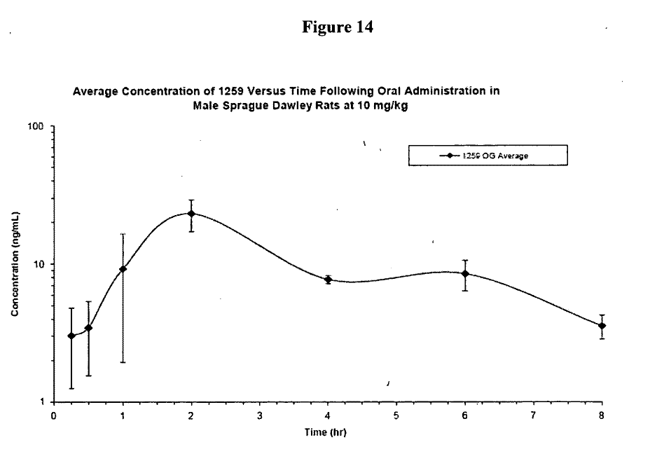

Data for the oral gavage of ICI-1259 is shown in Table 1. This data is represented graphically in FIG. 14. Following oral administration, the average bioavailability was 14%. This value indirectly indicates that approximately 38% of the dose is absorbed after oral dosing.

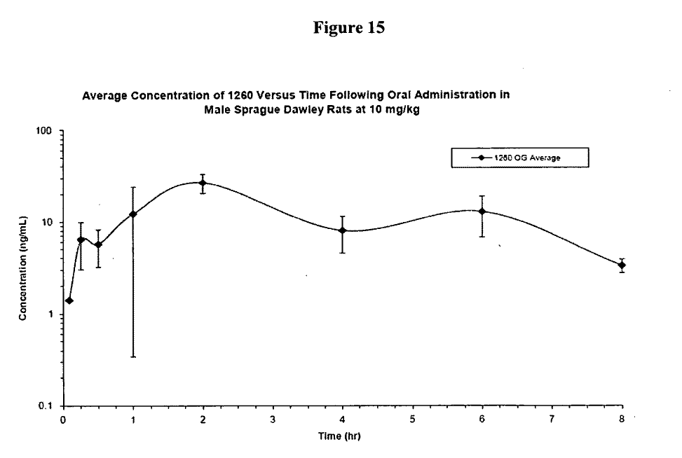

Data for the oral gavage of ICI-1260 is shown in Table 2. This data is represented graphically in FIG. 15. Following oral administration, the average bioavailability was 11%. This value indirectly indicates that approximately 26% of the dose is absorbed after oral dosing.

TABLE 1
Individual and Average Plasma Concentrations
(ng/mL) and Pharmacokinetic Parameters for
1259 After Oral Gavage Administration in Male Sprague-Dawley Rats
Oral Gavage (10 mg/kg)

| | Rat # | | | | |
|---|---|---|---|---|---|
| Time (hr) | 330 | 331 | 332 | Mean | SD |
| 0 (pre-dose) | BLOQ | BLOQ | BLOQ | ND | ND |
| 0.083 | BLOQ | BLOQ | BLOQ | ND | ND |
| 0.25 | 1.10 | 3.38 | 4.62 | 3.03 | 1.79 |
| 0.50 | 1.56 | 3.45 | 5.39 | 3.47 | 1.92 |
| 1.0 | 5.21 | 17.6 | 4.81 | 9.21 | 7.27 |
| 2.0 | 16.6 | 28.4 | 24.3 | 23.1 | 5.99 |
| 4.0 | 8.31 | 7.49 | 7.34 | 7.71 | 0.52 |
| 6.0 | 7.85 | 10.8 | 6.73 | 8.46 | 2.10 |
| 8.0 | 3.28 | 4.35 | 3.04 | 3.56 | 0.70 |
| Animal Weight (kg) | 0.304 | 0.288 | 0.301 | 0.298 | 0.009 |
| Volume Dosed (mL) | 3.040 | 2.880 | 3.010 | 2.977 | 0.085 |
| Cₘₐₓ (ng/mL) | 16.6 | 28.4 | 24.3 | 23.1 | 5.99 |
| tₘₐₓ (hr) | 2.0 | 2.0 | 2.0 | 2.0 | 0 |
| t_{1/2} (hr) | ND | ND | ND | ND | ND |
| AUCₗₐₛₜ (hr · ng/mL) | 65.2 | 98.7 | 74.2 | 79.4 | 17.3 |
| AUC_{∞} (hr · ng/mL) | ND | ND | ND | ND | ND |
| Dose Normalized | | | | | |
| Values² | | | | | |
| AUCₗₐₛₜ (hr · kg · | 6.52 | 9.87 | 7.42 | 7.94 | 1.73 |
| ng/mL/mg) | | | | | |
| AUC_{∞} (hr · kg · | ND | ND | ND | ND | ND |
| ng/mL/mg) | | | | | |
| Bioavailability (%)¹ | 11 | 17 | 13 | 14 | 3.0 |

Cₘₐₓ: Maximum plasma concentration;
tₘₐₓ: Time of maximum plasma concentration;
t_{1/2}: half-life, timepoints used in the half-life calculation are in bold;
AUCₗₐₛₜ: Area Under the Curve, calculated to the last observable timepoint;
AUC_{∞}: Area Under the Curve, extrapolated to infinity;
ND: Not Determined;
BLOQ: Below the limit of quantitation (1 ng/mL);
¹Bioavailability was determined by dividing the individual dose normalized AUCₗₐₛₜ values by the average intravenous AUCₗₐₛₜ value in Table 11;
²Dose normalized values determined by dividing the appropriate parameter by the nominal dosing concentration.

TABLE 2
Individual and Average Plasma Concentrations (ng/mL)
and Pharmacokinetic Parameters for
1260 After Oral Gavage Administration in Male Sprague-Dawley Rats
Oral Gavage (10 mg/kg)

| | Rat # | | | | |
|---|---|---|---|---|---|
| Time (hr) | 342 | 343 | 344 | Mean | SD |
| 0 (pre-dose) | BLOQ | BLOQ | BLOQ | ND | ND |
| 0.083 | 1.48 | 1.33 | BLOQ | 1.41 | ND |
| 0.25 | 9.32 | 2.68 | 7.31 | 6.44 | 3.41 |
| 0.50 | 8.00 | 3.02 | 6.16 | 5.73 | 2.52 |
| 1.0 | 5.33 | 5.38 | 25.9 | 12.2 | 11.9 |
| 2.0 | 33.8 | 22.2 | 24.0 | 26.7 | 6.24 |
| 4.0 | 4.89 | 11.7 | 7.39 | 7.99 | 3.44 |
| 6.0 | 19.8 | 9.71 | 8.98 | 12.8 | 6.05 |
| 8.0 | 3.13 | 2.87 | 3.93 | 3.31 | 0.55 |
| Animal Weight (kg) | 0.288 | 0.297 | 0.294 | 0.293 | 0.005 |
| Volume Dosed (mL) | 2.88 | 2.97 | 2.94 | 2.93 | 0.05 |
| Cₘₐₓ (ng/mL) | 33.8 | 22.2 | 25.9 | 27.3 | 5.93 |
| tₘₐₓ (hr) | 2.0 | 2.0 | 1.0 | 1.7 | 0.6 |
| t_{1/2} (hr) | ND | 2.2 | 2.6 | 2.4 | ND |
| AUCₗₐₛₜ (hr · ng/mL) | 112 | 84.9 | 95.9 | 97.7 | 13.8 |
| AUC_{∞} (hr · ng/ml) | ND | 94.0 | 111 | 102 | ND |
| Dose Normalized | | | | | |
| Values² | | | | | |
| AUCₗₐₛₜ (hr · kg · | 11.2 | 8.49 | 9.59 | 9.77 | 1.38 |
| ng/mL/mg) | | | | | |
| AUC_{∞} (hr · kg · | ND | 9.40 | 11.1 | 10.2 | ND |
| ng/mL/mg) | | | | | |
| Bioavailability (%)¹ | 13 | 9.0 | 11 | 11 | 2.1 |

Cₘₐₓ: Maximum plasma concentration;
tₘₐₓ: Time of maximum plasma concentration;
t_{1/2}: half-life, timepoints used in the half-life calculation are in bold;
AUCₗₐₛₜ: Area Under the Curve, calculated to the last observable timepoint;
AUC_{∞}: Area Under the Curve, extrapolated to infinity;
ND: Not Determined;
BLOQ: Below the limit of quantitation (1 ng/mL);
¹Bioavailability was determined by dividing the individual dose normalized AUC_{∞} values by the average intravenous AUC_{∞} value in Table 15, AUCₗₐₛₜ values used if no AUC_{∞} available;
²Dose normalized values determined by dividing the appropriate parameter by the nominal dosing concentration

### Collagen-Induced Arthritis Assay

Female Lewis rats (CR) weighing between 125-200 g were immunized with bovine type 2 collagen, 2 mg/ml in a 1:1 solution of 0.01 N acetic acid and Freund's incomplete adjuvant (“FIA”). A 0.1 ml portion of the collagen solution was injected id (intradermally) at three sites on the rat. Rats were immunized with the collagen solution on days 1 and 6 of the study. On days 7 and 8 arthritis was observed in the rats. On day 9 (study day “0”), baseline ankle diameters were measured. On days 10-15 (study days 1-6), the rats were then treated, via oral gavage, with either 1, 5, or 25 mg/kg of one of ICI-1259 or ICI-1260, prepared as described below. Ankle diameter measurements were measured on each of days 10-16.

Separate solutions of ICI-1259 and ICI-1260 were prepared in Dulbecco's PBS (pH 7) (“vehicle”) at 1 mg/ml. Control animals, who had not been immunized, were given vehicle without any compound. In a further control, immunized rats were given vehicle without ICI-1259 or ICI-1260. In still another control, immunized rats were treated with Enbrel™ at 10 mg/kg.

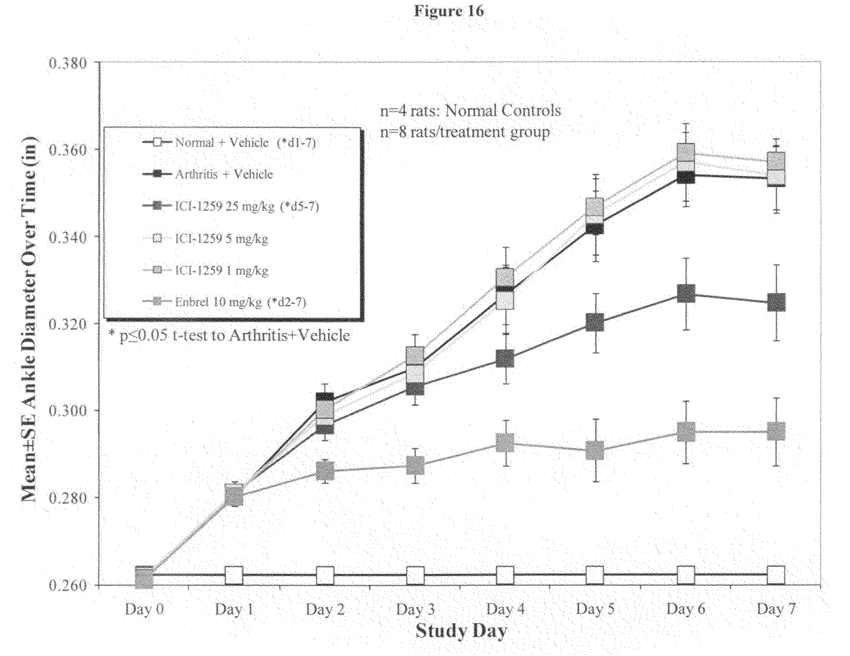

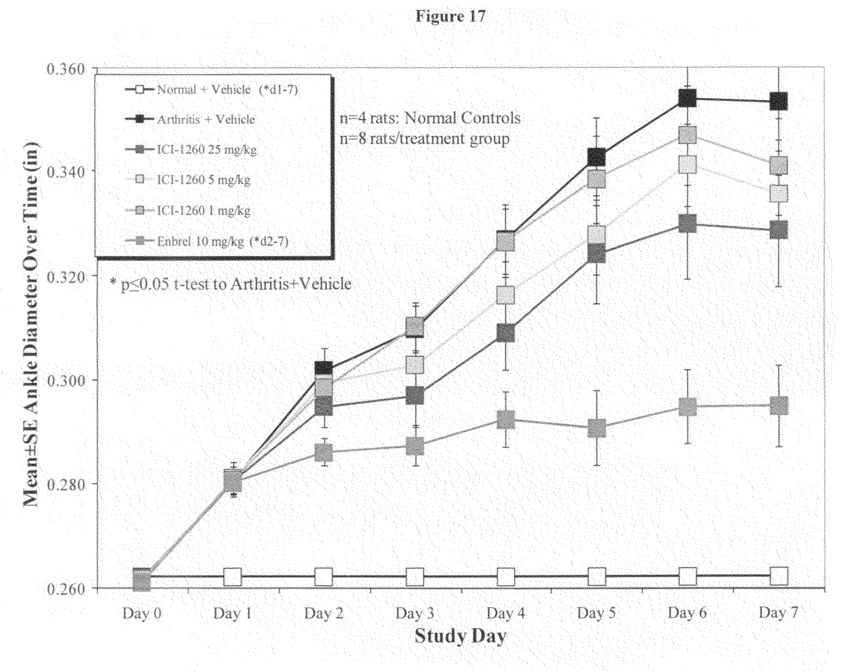

The results of the studies with ICI-1259 and ICI-1260 are shown in FIGS. 16 and 17, respectively. FIG. 16 shows that oral ICI-1259 at 25 mg/kg showed statistically significant reduction in ankle swelling over study days 5-7. FIG. 17 shows that oral ICI-1260 showed a reduction in ankle swelling at 5 and 25 mg/kg respectively, but that this compound had larger standard errors at each dose.

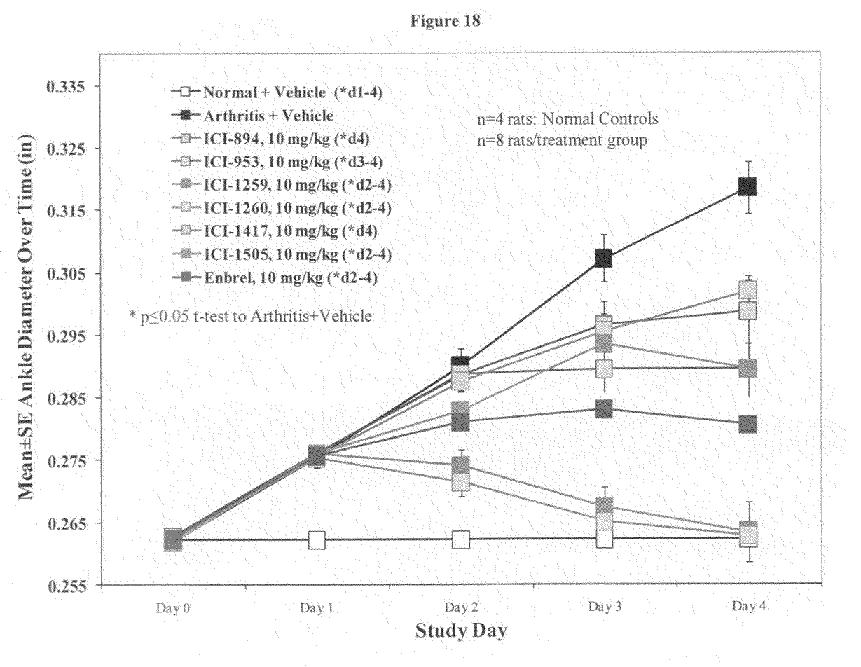

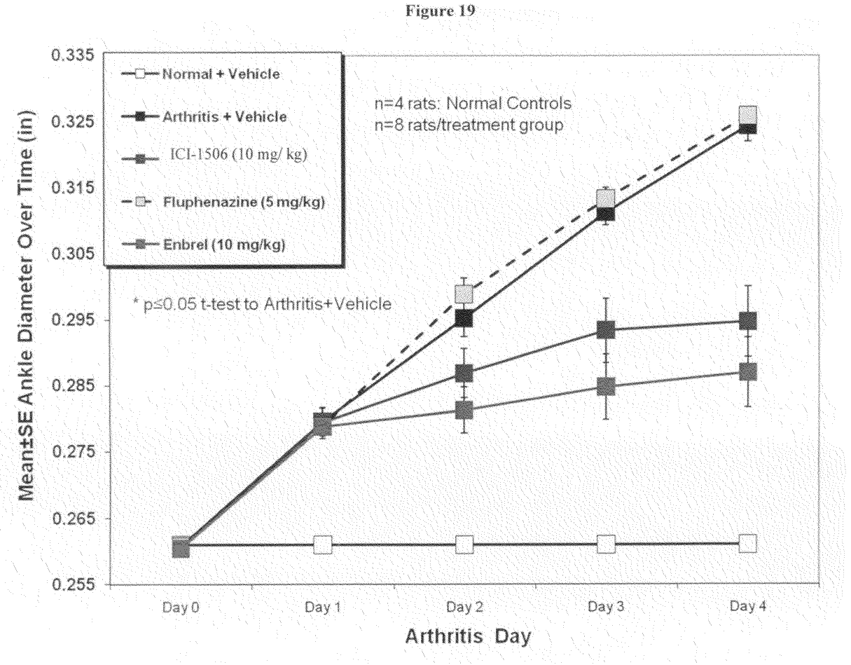

Compounds ICI-1259, ICI-1260, ICI-1505, and ICI-1506 were administered via IP injection to treat collagent-induced arthritis in a variation of the experiments described above. Under the protocol of the IP injection experiments, animal were immunized with Bovine Type 2 collagen as previously described. On days 7 and 8 arthritis was observed in the rats. On day 9 (study day “0”), baseline ankle diameters were measured. On days 10-12 (study days 1-3), the rats were then treated once per day, via IP injection, with one of ICI-1259, ICI-1260, ICI-1505, or ICI-1506 at 10 mg/kg. Ankle diameter in the immunized rodents was measured on each of days 10-13 as well as on day 14. For each compound, statistically significant results were observed on study days 2-4. See FIG. 18 (ICI-1259, ICI-1260, and ICI-1505) and FIG. 19 (ICI-1506).

In a separate study, using the IP injection protocol described above, ICI-1259 and 1260 were each dosed at 2 mg/kg and 0.4 mg/kg. Statistically significant results were only observed for ICI-1260 at 2 mg/kg on study days 3 and 4.

### 5-HT Receptor Binding

5-HT receptor binding was measured using membrane preparations of brain regions known to be enriched with given 5-HT receptor subtype(s). The assay subsequently measured the displacement of tritiated ligands known to bind to a given 5-HT subtype.

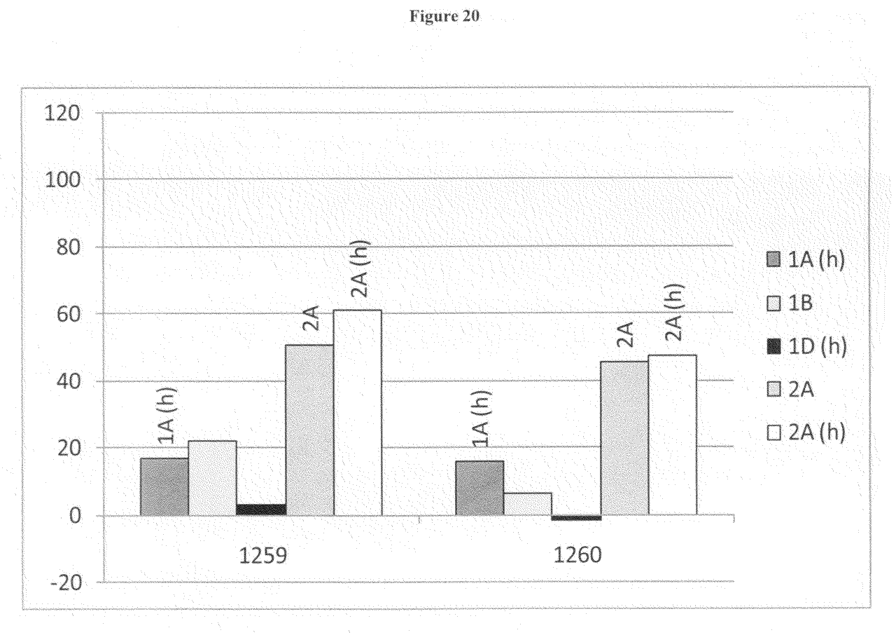

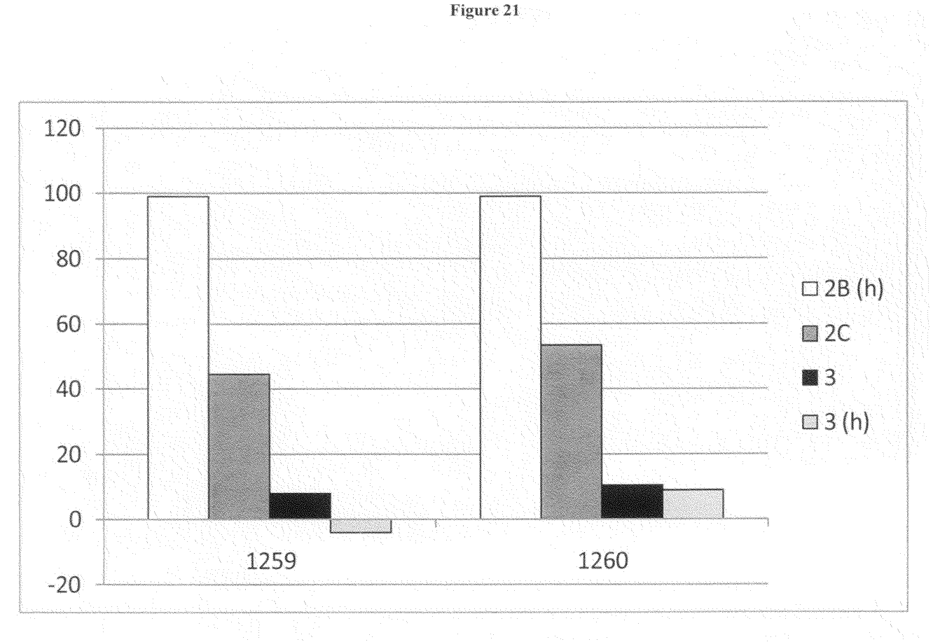

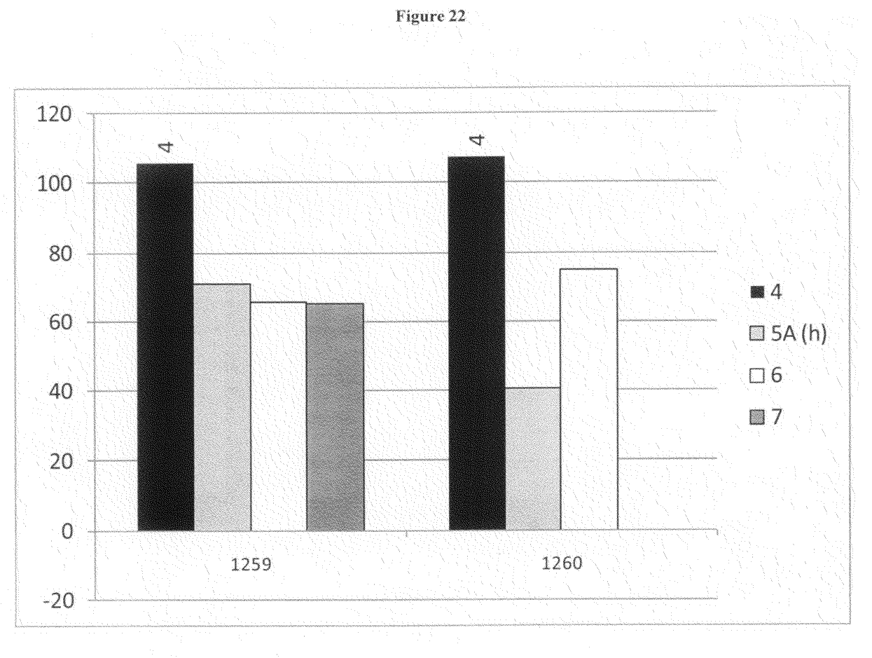

A receptor binding assay was performed for ICI-1259 and ICI-1260 showing that at 10 μM, these compounds bind receptors 5-HT1A(h), 5-HT1B, 5-HT1D(h), 5-HT2A, 5-HT2A(h), 5-HT2B, 5-HT2C, 5-HT3,5-HT3(h), 5-HT4, 5-HT5A(h), 5-HT6, and 5-HT7. See FIGS. 20-22.

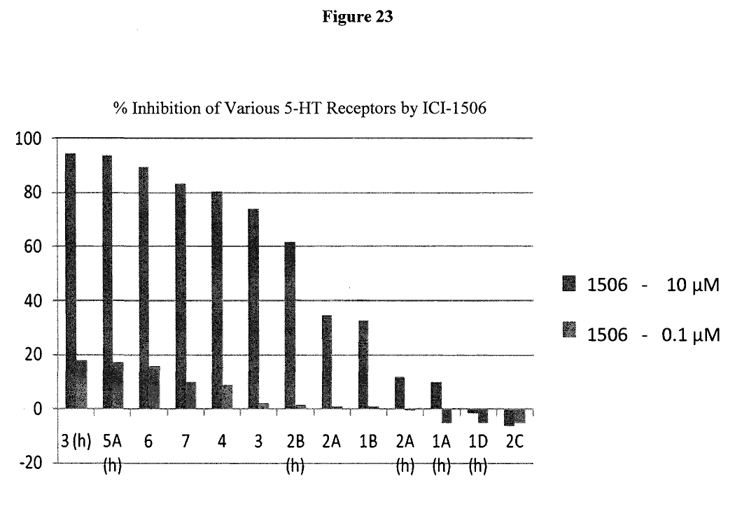

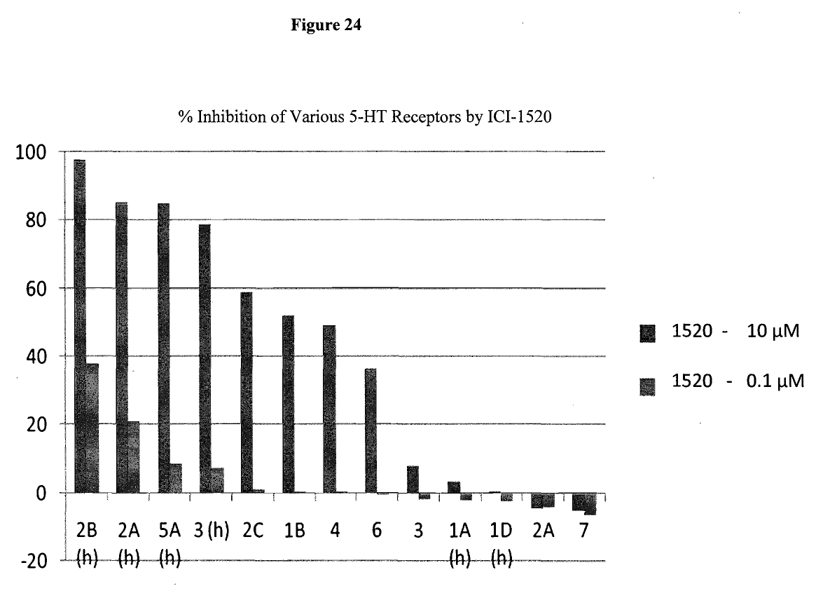

In a separate assays, the percent inhibition of various 5-HT receptors was measured with ICI-1506. See FIG. 23. A similar analysis was performed for ICI-1520. See FIG. 24. In both studies, the percent inhibition of the various 5-HT receptors was measured at 10 and 0.1 μM.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A compound of formula I 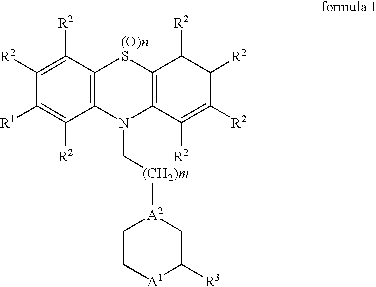
or a pharmaceutically acceptable salt, prodrug or solvate thereof, wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂;
NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is NR⁴;
A² is CH or N;
R⁴ is (CH₂)ₚR⁵; C(═O)(CH₂)ₚR⁵; or C(═O)(CH₂)ₚNR⁶₂;
R⁵ is 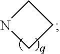
R⁶ is independently selected at each occurrence from the group consisting of hydrogen and (C₁-C₆)alkyl;
R⁷ is independently selected at each occurrence from the group consisting of hydrogen and (C₁-C₆)alkyl;
m is independently at each occurrence 1, 2, or 3;
n is 0, 1, or 2;
p is independently at each occurrence 2 or 3; and
q is independently at each occurrence 1 or 2.

2. The compound of claim 1, wherein: R¹ is hydrogen, halogen, (C₁-C₆)alkyl, methyl, C≡N, C(═O)OR⁷, C(═O)NR⁷₂, C(═O)NH₂, SO₂NR⁷₂, SO₂NMe₂, (C₁-C₃)perfluoroalkyl, or CF₃.

3. The compound of claim 1, wherein each occurrence of R² is hydrogen.

4. The compound of claim 1, wherein R³ is hydrogen.

5. The compound of claim 1, wherein A² is CH.

6. The compound of claim 1, wherein R⁴ is (CH₂)ₚR⁵.

7. The compound of claim 1, wherein R⁴ is C(═O)(CH₂)ₚR⁵.

8. The compound of claim 1, wherein R⁴ is C(═O)(CH₂)ₚNR⁶₂.

9. The compound of claim 7, wherein m is 2, n is 0, p is 2, and q is 1.

10. The compound of claim 6, wherein m is 2, n is 0, p is 2, and q is 3.

11. The compound of claim 1, wherein said compound is selected from the group consisting of ICI-1506, ICI-1520, ICI-1551, and ICI-1552.

12. A compound selected from the group consisting of ICI-955, ICI-956, ICI-957, ICI-1247, ICI-1259, ICI-1260, ICI-1451, and ICI-1505.

13. A method of inducing apoptosis in an immune cell, the method comprising contacting the immune cell with a composition comprising a compound of formula I or a composition comprising a compound selected from the group consisting of ICI-955, ICI-956, ICI-957, ICI-1247, ICI-1259, ICI-1260, ICI-1451, and ICI-1505;
wherein a compound of formula I has the structure: 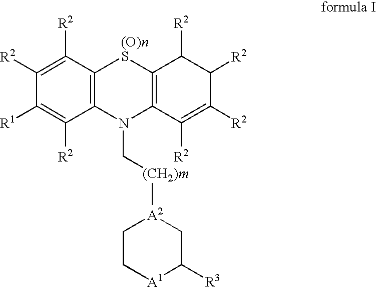
and wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is NR⁴;
A² is CH or N;
R⁴ is (CH₂)ₚR⁵; C(═O)(CH₂)ₚR⁵; or C(═O)(CH₂)ₚNR⁶₂;
R⁵ is 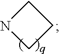
R⁶ is independently selected at each occurrence from the group consisting of hydrogen and (C₁-C₆)alkyl;
R⁷ is independently selected at each occurrence from the group consisting of hydrogen and (C₁-C₆)alkyl;
m is independently at each occurrence 1, 2, or 3;
n is 0, 1, or 2;
p is independently at each occurrence 2 or 3; and
q is independently at each occurrence 1 or 2.

14. The method of claim 13 wherein the immune cell is a lymphocyte.

15. The method of claim 14, wherein the lymphocyte is selected from the group consisting of a T cell and a B cell.

16. The method of claim 15, wherein the B cell is a plasma cell.

17. The method of claim 16 wherein, the plasma cell is a multiple myeloma cell.

18. A method of inhibiting proliferation of a lymphocyte, the method comprising contacting the lymphocyte with a composition comprising a compound of formula I or a composition comprising a compound selected from the group consisting of ICI-955, ICI-956, ICI-957, ICI-1247, ICI-1259, ICI-1260, ICI-1451, and ICI-1505;
wherein a compound of formula I has the structure: 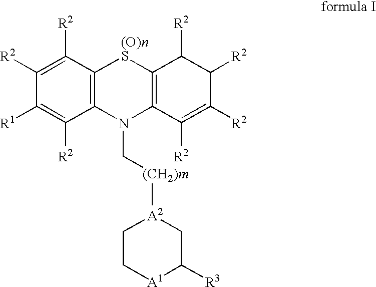
and wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C=1N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is NR⁴;
A² is CH or N;
R⁴ is (CH₂)ₚR⁵; C(═O)(CH₂)ₚR⁵; or C(═O)(CH₂)ₚNR⁶₂;
R⁵ is 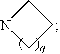
R⁶ is independently selected at each occurrence from the group consisting of hydrogen and (C₁-C₆)alkyl;
R⁷ is independently selected at each occurrence from the group consisting of hydrogen and (C₁-C₆)alkyl;
m is independently at each occurrence 1, 2, or 3;
n is 0, 1, or 2;
p is independently at each occurrence 2 or 3; and
q is independently at each occurrence 1 or 2.

19. The method of claim 18, wherein the lymphocyte is selected from the group consisting of a T cell and a B cell.

20. The method of claim 19, wherein the B cell is a plasma cell.

21. The method of claim 20, wherein the plasma cell is a multiple myeloma cell.

22. A method of treating a disease characterized by abnormal lymphocyte proliferation, the method comprising administering to a mammal a composition comprising a compound of formula I or a composition comprising a compound selected from the group consisting of ICI-955, ICI-956, ICI-957, ICI-1247, ICI-1259, ICI-1260, ICI-1451, and ICI-1505;
wherein a compound of formula I has the structure: 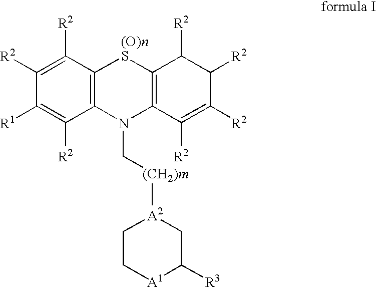
and wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂;
(C₂-C₆)alkylene-OR⁷; and (C₁-C₃)perfluoroalkyl;
R³ is hydrogen, C(═O)OR⁷, or C(═O)NR⁷₂;
A¹ is NR⁴;
A² is CH or N;
R⁴ is (CH₂)ₚR⁵; C(═O)(CH₂)ₚR⁵; or C(═O)(CH₂)ₚNR⁶₂;
R⁵ is 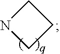
R⁶ is independently selected at each occurrence from the group consisting of hydrogen and (C₁-C₆)alkyl;
R⁷ is independently selected at each occurrence from the group consisting of hydrogen and (C₁-C₆)alkyl;
m is independently at each occurrence 1, 2, or 3;
n is 0, 1, or 2;
p is independently at each occurrence 2 or 3; and
q is independently at each occurrence 1 or 2.

23. A method of treating a disease selected from the group consisting of asthma and rheumatoid arthritis, the method comprising administering to a mammal a composition comprising a compound of formula I or a composition comprising a compound selected from the group consisting of ICI-955, ICI-956, ICI-957, ICI-1247, ICI-1259, ICI-1260, ICI-1451, and ICI-1505;
wherein a compound of formula I has the structure: 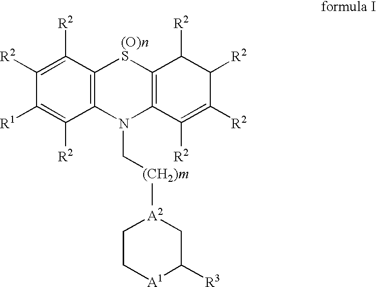
and wherein:
R¹ is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R² is independently selected at each occurrence from hydrogen, halogen, (C₁-C₆)alkyl; (C₁-C₆)alkenyl; (C₁-C₆)alkoxy; OH; NO₂; C≡N; C(═O)OR⁷; C(═O)NR⁷₂; NR⁷₂; NR⁷C(═O)(C₁-C₆)alkyl; NR⁷C(═O)O(C₁-C₆)alkyl; NR⁷C(═O)NR⁷₂; NR⁷SO₂(C₁-C₆)alkyl; SO₂NR⁷₂; OC(═O)(C₁-C₆)alkyl; O(C₂-C₆)alkylene-NR⁷₂; (C₂-C₆)alkylene-OR⁷; and (C₁-C₃)perfluoroalkyl;
R³ is hydrogen, C(═O)OR⁷, or C(═O)NR⁷₂;
A¹ is NR⁴;
A² is CH or N;
R⁴ is (CH₂)ₚR⁵; C(═O)(CH₂)ₚR⁵; or C(═O)(CH₂)ₚNR⁶₂;
R⁵ is 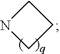
R⁶ is independently selected at each occurrence from the group consisting of hydrogen and (C₁-C₆)alkyl;
R⁷ is independently selected at each occurrence from the group consisting of hydrogen and (C₁-C₆)alkyl;
m is independently at each occurrence 1, 2, or 3;
n is 0, 1, or 2;
p is independently at each occurrence 2 or 3; and
q is independently at each occurrence 1 or 2.

24. The method of claim 23, wherein the mammal is a human.
